(19) United States
(12) Patent Application Publication
(10) Pub. No.: US 2023/0148910 A1
(43) Pub. Date: May 18, 2023

(54) LIFESTYLE ACTIVITY DETECTION FOR DIABETES MANAGEMENT

(71) Applicant: Dianavi Corporation, Irvine, CA (US)

(72) Inventors: Nicholas Hu, Irvine, CA (US); Tom Cao, Irvine, CA (US); Grace Qiao, Irvine, CA (US)

(21) Appl. No.: 17/914,988

(22) PCT Filed: Mar. 29, 2021
(86) PCT No.: PCT/US2021/024575

§ 371 Date: Sep. 27, 2022

Related U.S. Application Data
(60) Provisional application No. 63/001,152, filed on Mar. 27, 2020.
(60) Provisional application No. 63/001,156, filed on Mar. 27, 2020.

(51) Int. Cl. A61B 5/11; G16H 20/30; A61B 5/024; A61B 5/00; A63B 24/00

(52) U.S. Cl. CPC A61B 5/1118; G16H 20/30; A61B 5/024; A61B 5/486; A63B 24/0062; A61B 2562/0219

(57) ABSTRACT

A lifestyle coaching system for lifestyle intervention in disease management is provided. The lifestyle coaching system detects a patient performing various lifestyle activities, such as a workout or stress-inducing activity, as part of determining and optimizing real time or near-real time coaching output that is displayed to a patient via a user device. The provided lifestyle coaching system is able to detect a start and end time of each of the patient's workouts despite receiving the patient's heart rate data at a low sampling rate from a fitness tracking device by combining the low sample rate heart rate data with secondary factors indicative of patient working out. The provided lifestyle coaching system is also able to detect when an elevated heart rate of a patient is due to stress rather than exercise.

### PRIORITY CLAIM

The present application claims priority to and the benefit of U.S. Provisional Application 63/001,152, filed Mar. 27, 2020, and U.S. Provisional Application 63/001,156, filed Mar. 27, 2020. The entirety of each is herein incorporated by reference.

### TECHNICAL FIELD

The present application relates generally to the detection of lifestyle activities. More specifically, the present application provides system and methods for detecting that an individual has performed a lifestyle activity from physiological data of the individual.

### BACKGROUND

Diabetes affects an estimated 415 million people, or nearly one in eleven adults, globally. Experts expect this number to increase by more than 50% in the next twenty years, leading to in excess of 640 million cases worldwide. Diabetes treatments cost the U.S. alone upwards of two billion dollars annually. About 90-95% of these people are afflicted with Type 2 diabetes, which arises due to the body's inability to produce and/or use enough insulin. Though researchers believe some genetic factors may contribute to its development, other known risk factors for Type 2 diabetes include excess weight and physical inactivity. If left untreated, Type 2 diabetes can lead to glaucoma, nerve damage (particularly in the extremities), renal damage, and heart failure. Despite its severity, there is currently no cure for Type 2 diabetes. Instead, it is often mitigated through the control of lifestyle factors such as weight management, nutrition, adequate sleep, and exercise.

Lifestyle intervention is an effective means of controlling, treating, and reversing diabetes. A patient's lifestyle comprises a series of daily choices and actions that may contribute to the state of the disease. Additionally, a patient's daily context can be indicative of behavior that contributes to the progression of diabetes. The daily choices, actions, and context may include information indicative of nutrition, physical activity, mood, sleep, stress, adherence to medication routines, adherence to self-monitoring routines, environment, pollution, location, schedule, sedentary periods, and the like. Physicians, health coaches, nutritionists, disease educators, and other providers of medical care can help establish targets and plans for their patients. However, these patients are on their own for the vast majority of the time. Lifestyle coaching effectiveness for diabetes management often comes down to how well a patient is able to adhere to lifestyle recommendations without having constant intervention from their medical care providers. Oftentimes, patients regress back to poor lifestyle choices that led to their diabetes onset. In other instances, the lack of constant coaching enables patients to more easily become complacent with their health, ignoring the recommendations from their medical care provider.

Another issue with known lifestyle intervention is that medical care providers do not have an accurate picture of a patient's lifestyle prior to and after intervention. Instead, medical care providers only have access to subjective, and potentially biased, patient self-reporting of their lifestyle. For instance, patients often underestimate the amount of food and overestimate the quality of food they consume. Additionally, many patients often overestimate the amount of activity or exercise they perform. This subjective, and potentially biased, information can affect the assessment and lifestyle recommends that are provided by a medical care professional. In some instances, lifestyle coaching and treatment planning may only address the issues reported by the patient and neglect to treat unreported patient behavior. Further, even if a patient provides an accurate report on their lifestyle, a medical provider's recommendations are often static based on the initial recommendations. This may frustrate some patients who prefer to receive recognition of their lifestyle changes, or lack thereof, through an update to their recommended lifestyle.

Additionally, typical lifestyle intervention focuses on providing care in accordance with evidence-based guidelines. However, these guidelines are typically based on general populations or representative cohorts. While treatments according to these guidelines are generally associated with positive improvements in outcome for the patient population as a whole, such general treatments are not always the most effective treatment for an individual patient. Stated differently, general guidelines are developed at the population level, but medicine is practiced at the individual level. For example, the study presented in Zeevi, et al., Personalized Nutrition by Prediction of Glycemic Responses, CELL, vol. 163, issue 5, pp. 1079-94 (Nov. 19, 2015) showed that, based on a study of 800 people, certain foods have weaker or stronger glycemic impacts than others, and a general guideline of which foods to avoid or choose can be established at the population level. The study also showed, however, that there was a high variability in responses of individuals to identical meals in that certain foods were very good for some people while they were very bad for other people. As such, for some people, following guidelines derived from population level averages would be counterproductive to their individual physiologies.

One typical way to help provide personalized lifestyle guidelines or recommendations is to base such guidelines or recommendations on collected data particular to an individual. For example, such collected data could include heart rate data or step count data collected from a typical fitness tracking device, an accelerometer of a user device (e.g., a smartphone), and/or a fitness tracking application operating on the user device. In some instances, however, the data may be collected from the typical fitness tracking device, accelerometer of the user device, and/or fitness tracking application operating on the user device at a low sampling rate that limits the ability to generate guidelines or recommendations from that data, or that limits the accuracy of generated guidelines or recommendations from that data. For example, typical fitness tracking devices will record a user's heart rate every 5 minutes to 10 minutes to conserve battery life, since a user's heart rate does not vary significantly during most of the day, unless a “record” setting is activated that increases the heart rate sample rate to about every five seconds. If the user forgets to activate the “record” setting when beginning to work out, the typical fitness tracking device will continue to collect data at a sample rate of 5 to 10 minute intervals, omitting significant exercise data.

Additionally or alternatively, the typical fitness tracking device or fitness tracking application on a user device may be limited in the types of personalized guidelines or recommendations that the typical fitness tracking device or user device can generate based on that collected data. For example, a typical fitness tracking device or fitness tracking application on a user device may incorrectly attribute an elevated heart to exercise when the elevated heart rate is really due to stress. Accordingly, in such examples, the typical fitness tracking device or fitness tracking application on a user device cannot provide personalized guidelines or recommendations related to the user's stress.

### SUMMARY

The present application provides new and innovative systems and methods for lifestyle intervention in disease management. The lifestyle coaching system factors patient context, behavior, and predicted patterns in addition to physiological data for determining and optimizing real time or near-real time coaching output that is displayed to a patient via a user device. In connection with determining and optimizing real time or near-real time coaching, the lifestyle coaching system may detect that the patient is performing or has performed various lifestyle activities. For example, the lifestyle coaching system may detect that the patient exercised (e.g., performed a workout) or that the patient performed a stressful activity based on physiological data (e.g., heart rate data, step data, etc.) of the patient received from a typical fitness tracking device, an accelerometer of the user device (e.g., a smartphone), and/or a fitness tracking application operating on the user device.

The provided lifestyle coaching system is able to detect a start and end time of each of the patient's workouts despite receiving the patient's heart rate data at a low sampling rate from a fitness tracking device by combining the low sample rate heart rate data with secondary factors indicative of the patient working out. The provided lifestyle coaching system is also able to detect when an elevated heart rate of a patient is due to stress rather than exercise. As the provided lifestyle coaching system collects data on the detected occurrences of the patient performing various lifestyle activities, such as working out or experiencing stress, this collected data may be correlated with other contextual information. For instance, the provided lifestyle coaching system may correlate a location with the patient experiencing stress and can then display a notification message on the user device alerting the patient that they are approaching a stressful location so that the patient may be cognizant of their stress level. In this way, the lifestyle coaching system can determine and optimize coaching output with regard to various lifestyle activities of the patient.

In light of the technical features set forth herein, and without limitation, in a first aspect of the disclosure in the present application, which may be combined with any other aspect unless specified otherwise, a system for detecting lifestyle activities includes a processor in communication with a memory. The processor is configured to receive user data including step count data of a user and/or heart rate data of the user over a period of time; determine at least one of: (i) a rate of steps in the received step count data is greater than a threshold step rate over at least a subset of the period of time, or (ii) the received heart rate data includes a heart rate above a threshold heart rate over at least a subset of the period of time; determine whether a secondary factor indicative of a first lifestyle activity is present in the user data during the period of time; if the secondary factor is not present, classify the period of time as a second lifestyle activity; determine whether a length of the period of time is greater than a first threshold period of time if the secondary factor is present; if the length of the period of time is less than the first threshold period of time, classify the period of time as the second lifestyle activity; if the length of the period of time is greater than the first threshold period of time, classify the period of time as the first lifestyle activity; and combine the period of time classified as the first lifestyle activity with a previous period of time classified as the first lifestyle activity in response to determining that less than a second threshold period of time has elapsed between the period of time classified as the first lifestyle activity and the previous period of time classified as the first lifestyle activity.

In a second aspect of the disclosure in the present application, which may be combined with any other aspect unless specified otherwise, the step count data of the user and/or the heart rate data of the user is received from a fitness tracking device, an accelerometer of a user device, and/or a fitness tracking application operating on a user device.

In a third aspect of the disclosure in the present application, which may be combined with any other aspect unless specified otherwise, the heart rate data is received at a lower sampling rate than the step count data is received.

In a fourth aspect of the disclosure in the present application, which may be combined with any other aspect unless specified otherwise, the heart rate data is received at a sampling rate of between every 5-10 minutes.

In a fifth aspect of the disclosure in the present application, which may be combined with any other aspect unless specified otherwise, the threshold heart rate is equal to a percentage of a maximum heart rate of the user.

In a sixth aspect of the disclosure in the present application, which may be combined with any other aspect unless specified otherwise, the threshold heart rate is equal to 57-63% of a maximum heart rate of the user.

In a seventh aspect of the disclosure in the present application, which may be combined with any other aspect unless specified otherwise, the secondary factor indicative of the first lifestyle activity is at least one of: (i) a heart rate of the user exceeding the threshold heart rate if it is determined that the rate of steps is greater than the threshold step rate, (ii) a location of the user being a location associated with the first lifestyle activity, (iii) a rate of location change of the user exceeding a threshold rate, and (iv) a step count greater than zero in the received step count data if it is determined that the received heart rate data includes a heart rate above the threshold heart rate.

In an eighth aspect of the disclosure in the present application, which may be combined with any other aspect unless specified otherwise, the secondary factor indicative of the first lifestyle activity is a heart rate of the user exceeding the threshold heart rate if it is determined that the rate of steps is greater than the threshold step rate.

In a ninth aspect of the disclosure in the present application, which may be combined with any other aspect unless specified otherwise, the secondary factor indicative of the first lifestyle activity is a location of the user being a location associated with the first lifestyle activity.

In a tenth aspect of the disclosure in the present application, which may be combined with any other aspect unless specified otherwise, the secondary factor indicative of the first lifestyle activity is a rate of location change of the user exceeding a threshold rate.

In an eleventh aspect of the disclosure in the present application, which may be combined with any other aspect unless specified otherwise, the secondary factor indicative of the first lifestyle activity is a step count greater than zero in the received step count data if it is determined that the received heart rate data includes a heart rate above the threshold heart rate.

In a twelfth aspect of the disclosure in the present application, which may be combined with any other aspect unless specified otherwise, the first lifestyle activity is working out.

In a thirteenth aspect of the disclosure in the present application, which may be combined with any other aspect unless specified otherwise, the second lifestyle activity is an activity other than working out.

In a fourteenth aspect of the disclosure in the present application, which may be combined with any other aspect unless specified otherwise, the processor is further configured to categorize an intensity of the period of time classified as the first lifestyle activity based on at least one of (i) an average heart rate, (ii) a median heart rate, (iii) a maximum heart rate, and (iv) a quantity of heart rate data points, of the received heart rate data in the period of time compared to a predetermined maximum heart rate up the user.

In a fifteenth aspect of the disclosure in the present application, which may be combined with any other aspect unless specified otherwise, the processor is further configured to determine a resting heart rate baseline of the user based on previously received heart rate data of the user.

In a sixteenth aspect of the disclosure in the present application, which may be combined with any other aspect unless specified otherwise, determining the resting heart rate baseline based on previously received heart rate data includes eliminating the heart rate data associated with periods of activity of the user from the previously received heart rate data.

In a seventeenth aspect of the disclosure in the present application, which may be combined with any other aspect unless specified otherwise, heart rate data associated with a predetermined amount of time directly prior to, and directly subsequent to, the periods of activity of the user are further eliminated from the previously received heart rate data.

In an eighteenth aspect of the disclosure in the present application, which may be combined with any other aspect unless specified otherwise, upon determining that the secondary factor is not present or that the length of the period of time is less than the first threshold period of time the processor is further configured to: determine whether the received heart rate data includes an average or median heart rate that exceeds the determined resting heart rate baseline, wherein if it is so determined, the second lifestyle activity is a stress-inducing activity, and wherein if it is not so determined, the second lifestyle activity is an activity other than working out and other than a stress-inducing activity.

In a nineteenth aspect of the disclosure in the present application, which may be combined with any other aspect unless specified otherwise, the processor is further configured to classify a severity of the stress induced by the stress-inducing activity based on the percentage of a standard deviation that an average of the received heart rate data is equal to.

In a twentieth aspect of the disclosure in the present application, which may be combined with any other aspect unless specified otherwise, a method for detecting lifestyle activities includes receiving, from a fitness tracking device, an accelerometer of a user device, and/or a fitness tracking application operating on a user device, user data including step count data of a user and/or heart rate data of the user over a period of time. From the received data, at least one of the following is determined: (i) a rate of steps in the received step count data is greater than a threshold step rate over at least a subset of the period of time, or (ii) the received heart rate data includes a heart rate above a threshold heart rate over at least a subset of the period of time. It is then determined whether a secondary factor indicative of a first lifestyle activity is present in the user data during the period of time. If the secondary factor is not present, the period of time is classified as a second lifestyle activity. If the secondary factor is present, it is determined whether a length of the period of time is greater than a first threshold period of time. If the length of the period of time is less than the first threshold period of time, the period of time is classified as the second lifestyle activity. If the length of the period of time is greater than the first threshold period of time, the period of time is classified as the first lifestyle activity. The period of time classified as the first lifestyle activity may then be combined with a previous period of time classified as the first lifestyle activity in response to determining that less than a second threshold period of time has elapsed between the period of time classified as the first lifestyle activity and the previous period of time classified as the first lifestyle activity.

In a twenty-first aspect of the disclosure in the present application, which may be combined with any other aspect unless specified otherwise, determining whether the length of the period of time is greater than the threshold period of time includes determining a termination point of the period of time.

In a twenty-second aspect of the disclosure in the present application, which may be combined with any other aspect unless specified otherwise, determining the termination point of the period of time includes at least one of: (i) determining that the rate of steps in the received step count data is no longer greater than the threshold step rate, (ii) determining that the received heart rate data no longer includes a heart rate above the threshold heart rate, (iii) receiving location data indicating that the user has left the location associated with the first lifestyle activity, and (iv) receiving location data indicating that the rate of location change of the user no longer exceeds the threshold rate.

In a twenty-third aspect of the disclosure in the present application, which may be combined with any other aspect unless specified otherwise, determining the termination point of the period of time includes determining that the rate of steps in the received step count data is no longer greater than the threshold step rate.

In a twenty-fourth aspect of the disclosure in the present application, which may be combined with any other aspect unless specified otherwise, determining the termination point of the period of time includes determining that the received heart rate data no longer includes a heart rate above the threshold heart rate.

In a twenty-fifth aspect of the disclosure in the present application, which may be combined with any other aspect unless specified otherwise, determining the termination point of the period of time includes receiving location data indicating that the user has left the location associated with the first lifestyle activity.

In a twenty-sixth aspect of the disclosure in the present application, which may be combined with any other aspect unless specified otherwise, determining the termination point of the period of time includes receiving location data indicating that the rate of location change of the user no longer exceeds the threshold rate.

In a twenty-seventh aspect of the disclosure in the present application, which may be combined with any other aspect unless specified otherwise, determining the termination point includes interpolating between a last heart rate data point that exceeds the threshold heart rate and a first heart rate data point that fails to exceed the threshold heart rate.

In a twenty-eighth aspect of the disclosure in the present application, which may be combined with any other aspect unless specified otherwise, determining a start of the period of time includes interpolating between a last heart rate data point that fails to exceed the threshold heart rate and a first heart rate data point that exceeds the threshold heart rate.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 shows a diagram of a diabetes coaching system, according to an example embodiment of the present disclosure.

FIG. 2 shows a diagram of a user device of the diabetes coaching system of FIG. 1, according to an example embodiment of the present disclosure.

FIG. 3 shows a diagram of an actionable recommendation for the diabetes coaching system of FIG. 1, according to an example embodiment of the present disclosure.

FIG. 4 shows a diagram of example types of patient data that may be received by an application and/or a diabetes management server for the diabetes coaching system of FIG. 1, according to an example embodiment of the present disclosure.

FIG. 5 shows a flow diagram of an example procedure for using patient data to detect a lifestyle activity of a patient, according to an aspect of the present disclosure.

FIG. 6 shows a flow diagram of an example procedure for using patient data to detect that a lifestyle activity of a patient is an activity inducing stress in the patient, according to an aspect of the present disclosure.

FIGS. 7 and 8 each show a user interface of the user device of FIG. 2 displaying an example graph of a distribution of heart rate data and step data for a patient over a portion of a day, according to an aspect of the present disclosure.

FIGS. 9 to 12 each show a user interface of the user device of FIG. 2 displaying a graph and table of a distribution over a time period of heart rate data received at a low sampling rate and of step data, from which it may be determined whether the time period is the first lifestyle activity, according to an aspect of the present disclosure.

FIG. 13 shows a user interface of the user device of FIG. 2 displaying an example collection of inactive heart rate data distributions that each show a frequency of a patient's inactive heartrate at a particular beats per minute (bpm) value over the course of a day, according to an aspect of the present disclosure.

FIG. 14 shows a user interface of the user device of FIG. 2 displaying an example collection of inactive heart rate data distributions that each show a frequency of a user's inactive heartrate at a particular bpm value at different parts of a single day, according to an aspect of the present disclosure.

FIG. 15 shows a user interface of the user device of FIG. 2 displaying an example graph of a comparison of a patient's heart rate data as a distribution over the past 1 day, past 7 days, and past 30 days, according to an aspect of the present disclosure.

FIG. 16 shows a user interface of the user device of FIG. 2 displaying an example graph showing a distribution of inactive heart rates over a single day with assigned stress scores, according to an aspect of the present disclosure.

FIG. 17 shows a user interface of the user device of FIG. 2 displaying a collection of data tables showing a patient's stress score data distributed from 10 AM to 10 PM in one hour time blocks across a different days, according to an aspect of the present disclosure.

FIG. 18 shows a user interface of the user device of FIG. 2 displaying an example stress heat map that shows a patient's stress score data distributed from 10 AM to 10 PM across each date in February, according to an aspect of the present disclosure.

FIG. 19 shows a user interface of the user device of FIG. 2 displaying an example stress heat map that shows a patient's stress score data distributed from 10 AM to 10 PM across multiple dates in March that are further categorized as days of the week, according to an aspect of the present disclosure.

FIG. 20A shows a user interface of the user device of FIG. 2 displaying a patient's stress data in the form of a calendar, according to an aspect of the present disclosure.

FIG. 20B shows a user interface of the user device of FIG. 2 displaying an hourly breakdown of the patient's stress for a particular day from the calendar of FIG. 20A, according to an aspect of the present disclosure.

FIG. 21 shows an example holistic model that correlates exercise duration and intensity with calories consumed, carbohydrates consumed, average blood glucose, and an amount of time the patient's blood glucose is in a desired range, according to an aspect of the present disclosure.

FIG. 22 shows a user interface of the user device of FIG. 2 displaying a graph that charts the patient's detected resting heart rate from just prior to March 2020 to just after January 2021, and a graph that charts the patient's detected exercise periods during the same time period, according to an aspect of the present disclosure.

FIG. 23A shows a user interface of the user device of FIG. 2 displaying statistics of a patient's workout data, according to an aspect of the present disclosure.

FIG. 23B shows a user interface of the user device of FIG. 2 displaying charts of the patient workout data, according to an aspect of the present disclosure.

FIG. 24 shows a user interface of the user device of FIG. 2 displaying a graph that charts data indicating how the patient spends their time at fourteen different locations, according to an aspect of the present disclosure.

FIG. 25 shows a user interface of the user device of FIG. 2 displaying an example map including example indicators at various locations, according to an aspect of the present disclosure.

FIG. 26 shows user interfaces of the user device of FIG. 2 displaying notification messages based on actionable recommendations, according to example embodiments of the present disclosure.

### DETAILED DESCRIPTION

The systems, methods, and apparatus disclosed herein are directed to automated lifestyle coaching based on patient context, physiology, behavior, and predicted patterns. Disclosed herein is a lifestyle coaching system (e.g., an application and a connected diabetes management server) that is configured for lifestyle intervention in disease management. The lifestyle coaching system factors patient context, behavior, and predicted patterns in addition to physiological data for determining and optimizing real time or near-real time coaching output that is displayed to a patient via a user device. The system may also factor relevant clinical guidelines for lifestyle management of the patient. It should be appreciated that the systems, methods, and apparatus provide coaching for patients with Type 2 diabetes, Type 1 diabetes, and/or gestational diabetes.

In an example, overweight, obese, pre-diabetic, and diabetic patients benefit from medical nutritional therapy. The present system is configured as an extension or replacement of a plan that is developed by a physician or nutritionist. The plan may prescribe certain caloric budgets, macronutrient breakdowns, and suggested food sources. For instance, a high-level lifestyle modification such as reducing carbohydrate intake could be established by a medical care provider with a suggestion that this could be met by avoiding grains and pastas. Alternatively, the systems, methods, and apparatus may determine the high-level lifestyle modification through an analysis of patient data. These suggestions or lifestyle modifications represent high-level coaching that patients may not necessarily understand how to implement throughout a patient's daily life.

The systems, methods, and apparatus disclosed herein help such patients by providing automated coaching to a patient that translates high-level lifestyle modifications into discrete actionable recommendations. The recommendations are triggered for display at contextual-appropriate times to increase a probability that the patient will follow the recommendation. In the food example above, the recommendation may include identification of certain food items on a menu of a particular restaurant that is in proximity to the patient during typical patient mealtimes. In this manner, the systems, methods, and apparatus disclosed herein transform lifestyle treatment goals into patient-specific actionable guidance that is triggered in real time or near-real time based on the patient's context, location, and/or eating pattern. The systems, methods, and apparatus disclosed herein provide more effective coaching by providing actionable coaching. In other words, the systems, methods, and apparatus disclosed herein factor patient goals against patient context and other lifestyle behaviors to identify immediate opportunities for diabetes health improvement.

In some embodiments, the systems, methods, and apparatus may be configured to use proxy indicators for automated health coaching. For example, the systems, methods, and apparatus are configured to determine that whenever a patient arrives at a specific GPS coordinate, the patient's blood sugar spikes fifteen minutes later. The systems, methods, and apparatus may then use this contextual information to coach the patient against visiting this location to prevent blood sugar spikes. In this example, the systems, methods, and apparatus uses GPS data (determined from the patient's user device) as a proxy biomarker for diabetes prevention or management.

In contrast to the systems, methods, and apparatus disclosed herein, traditional lifestyle coaching focuses on identifying the food source that causes a rise in blood sugar, which could cause diabetic complications. A medical care provider then provides a written recommendation to the patient to avoid the identified food source. However, this traditional approach requires accurate self-reporting by the patient. Even if the food source is accurately identified, the recommendation becomes stale or forgotten by the patient as time passes. This may be especially true when the patient is tempted by the food source such that the recommendations are at least temporally mentally blocked, suppressed, or rationalized.

The example systems, methods, and apparatus provide a technical innovation over traditional lifestyle coaching by providing actionable lifestyle recommendations that are triggered based on the patient's current contextual disposition. The systems, methods, and apparatus disclosed herein may coach a patient to simply avoid a certain location. In this instance, the recommendation may not necessarily identify what food source at that location causes a blood sugar spike. However, the systems, methods, and apparatus provide the same avoidance benefit through the automated recommendation for avoiding diabetic complications.

In another example, the systems, methods, and apparatus are configured to determine that a patient's calendar is very busy on a certain day around lunchtime. Given that this patient may be an insulin dependent diabetic, who is at risk of hypoglycemic events, the systems, methods, and apparatus may provide a recommendation or guidance to the patient on that morning to bring a snack with high carbohydrate content. Further, after detecting that the current time is at or around noon, the systems, methods, and apparatus provide a recommendation to eat the snack. In this manner, the systems, methods, and apparatus have automatically coached the patient in real time or near-real time using contextual information such as the patient's schedule (and granular blood sugar history) as a biomarker proxy to manage diabetes.

As described herein, the systems, methods, and apparatus enhance coaching effectiveness by providing more timely output. For example, elderly diabetic patients benefit from flexibility and balance training. The present systems, methods, and apparatus are configured to consider a patient's age and diabetic state when determining whether it is in the patient's best interest to undertake at least three flexibility or balance training sessions per week. To accomplish this, the example the systems, methods, and apparatus may provide a recommendation at the beginning of a week to perform three flexibility or balance sessions. Additionally, during the week, the systems, methods, and apparatus analyze patient data for indications as to whether a flexibility or balance session was performed. If at least one session was not performed, the systems, methods, and apparatus transmit reminder notification messages to the patient. The reminders are generated at days/times corresponding to when the patient has previously performed a flexibility or balance session and/or days/times corresponding to patient inactivity.

In some embodiments, the systems, methods, and apparatus generate a preset message at a scheduled day/time unless an event happens or does not happen. For example, a reminder to perform flexibility training can be scheduled for display to a patient on a Friday unless at least three training sessions were performed during the week. If the patient has already performed three of the three training sessions by Thursday of that week, the systems, methods, and apparatus refrains from creating the notification. Instead, the systems, methods, and apparatus may generate a congratulatory message or provide some sort of electronic recognition that the patient is providing a positive impact on their diabetic health by following the recommendations. This feedback provides the patient with a positive affirmation that the systems, methods, and apparatus are specifically adapted to their lifestyle instead of being based on a generic template.

Another method of providing coaching is to predict a coaching output that may be necessary for some period in the future, and schedule the coaching in advance. If any relevant patient data is received between initial scheduling and the scheduled output time, the systems, methods, and apparatus may reevaluate the existing prescheduled coaching output and cancel, edit, reschedule, and/or add additional coaching as warranted by patient status changes. For example, patients may need to perform three sessions of weight training per week without performing weight training on consecutive days. On Monday, the start of the week, the systems, methods, and apparatus schedule a message for Wednesday that recommends the patient perform a weight training session that day to be on track to achieve three sessions this week. If the patient then proceeds to perform a weight training session on Tuesday, the originally scheduled message is cancelled and a new message is scheduled for Friday that recommends the patient perform a weight training session that day to achieve three sessions this week. The systems, methods, and apparatus are configured with this predictive coaching as needed by the patient, and update planned notifications based on received patient data.

Reference is made herein to patient data. As disclosed herein, patient data includes contextual, physiological, and/or behavioral data related to the lifestyle of a patient. The patient data may include age, sex, height, weight, diabetes type, medication schedule, metabolic data, heart rate, blood pressure, body temperature, electrocardiogram data, respiratory rate, reproductive cycle, blood glucose level, A1C results, insulin intake, mood, mindfulness, exercise information, time awake/sleep, nutritional intake data, nutritional source data, patient location, time of day, weather, pollution level, etc. In other words, the patient data provides indicative of a past, current, or predicted disposition of a patient. As disclosed herein, the patient data may be received from a sensor or other connected device, self-reported by the patient, or received from a third-party server or application.

### Diabetes Coaching System Embodiment

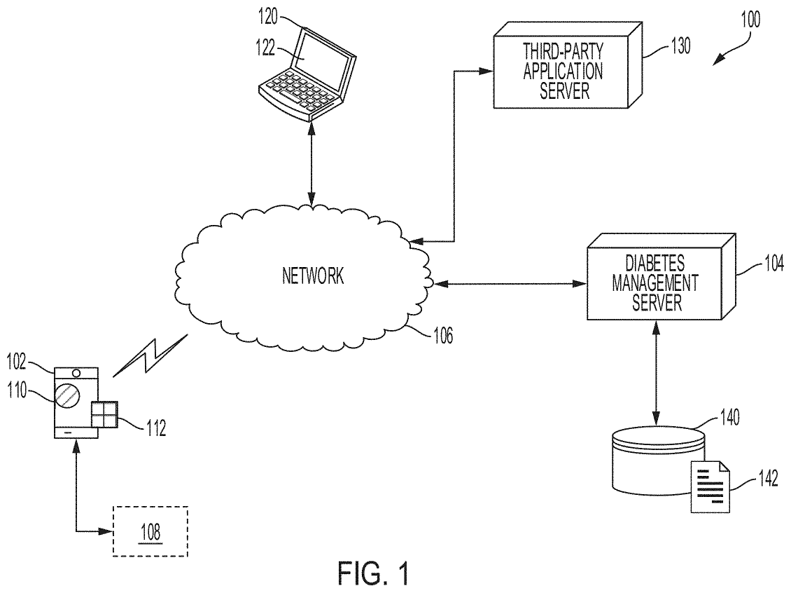

FIG. 1 shows a diagram of a diabetes coaching system 100, according to an example embodiment of the present disclosure. The example diabetes coaching system 100 includes a user device 102 configured to record and/or receive patient data. The system 100 also includes a diabetes management server 104 configured to receive at least some of the patient data from the user device 102 for analysis and/or lifestyle coaching. The user device 102 is connected to the diabetes management server 104 via a network 106.

The example user device 102 may include a smartphone, cellular phone, tablet computer, laptop computer, personal computer, workstation, smartwatch, smart-eyewear, etc. The diabetes management server 104 may include a processor, a group of processors, a controller, a microcontroller, a database etc. for receiving/storing data, performing computations, and outputting data. The network 106 may include any wired and/or wireless network including the Internet, an Ethernet, a cellular network, or combinations thereof.

In the illustrated example, the user device 102 is communicatively coupled to an external sensor device 108, which may be included in, for example, a fitness tracking device or bracelet. For example, the sensor device 108 may be included in a fitness tracker from Fitbit, Inc. The external sensor device 108 may include one or more physiological sensors and is configured to measure physiological and/or patient data including heartbeat data, weight data, blood pressure data, a number of steps taken data, a pace of steps taken data, breathing data, GPS data, glucose level data, sleep state data, etc. The user device 102 may be wired or wirelessly coupled to the sensor device 108 via, for example, a USB® connection, a Bluetooth® connection, a Lightning® connection, an NFC connection, etc.

While FIG. 1 shows only a single user device 102, it should be appreciated that the system 100 may include additional user devices. For example, the diabetes management server 104 may be in communication with thousands or millions of user devices for receiving respective patient data from patients and performing automated diabetes health coaching. In this example, the diabetes management server 104 transmits personalized health recommendations for each of the patients at times that are likely to improve adherence to, for example, a diabetes management plan.

The example user device 102 of FIG. 1 includes an application 110 and a processor 112. The application 110 is defined by or specified by one or more machine-readable instructions stored in a memory device of the user device 102. The instructions may specify one or more algorithms, routines, or operations performed by the application 110. The instructions may also specify one or more user interfaces for display on a screen of the user device 102. The processor 112 executes the instructions to provide for operation of the application 110. Disclosure herein to the application 110 performing certain operations refers to the instructions executed by the processor 112 to enable the operations to be carried out on the user device 102. The application 110 may include a stand-alone software application (e.g., an app), a web browser, or a plug-in for a web browser. In some embodiments, the application 110 may operate without connecting to the server 104, or even if the server 104 is not present in the system 100.

In addition to the user device 102, the example diabetes coaching system 100 of FIG. 1 includes one or more clinician devices 120. The clinician device 120 includes any smartphone, tablet computer, laptop computer, desktop computer, smart watch, smart-eyewear, server, etc. to enable a clinician to view and/or provide comments or make recommendations for a patient. This may include the clinician creating a diabetes management plan that provides high-level lifestyle modifications. The clinician device 120 may include a clinician application 122 configured to provide one or more user interfaces for viewing a patient's data, a patient's health plan, and/or determined actionable recommendations. The application 122 may be communicatively coupled to one or more APIs at the diabetes management server 104 for providing (e.g., downloading/uploading) patient data and/or timeline information to enable the application 122 to display a graphical timeline of a patient's recommendations and/or lifestyle activities. The application 122 may also include one or more user interfaces to view recommendations generated by the server 104 that have been transmitted, or awaiting transmission, to a user device 102 of a patient. In some instances, the application 122 in combination with the diabetes management server 104 are configured to have a clinician approve a recommendation (or certain types of recommendations related to insulin administration, significant activity changes, etc.) before it is transmitted to the user device 102. The application 122 may also enable a clinician to create and/or modify recommendations for a patient after viewing their data and/or recorded activities. The recommendations are transmitted by the application 122 to the diabetes management server 104, which then transmits the recommendations to the user device 102 in one or more notification messages at designated times.

In some embodiments, the diabetes coaching system 100 includes a third-party server 130. As disclosed herein, the third-party server 130 is configured to provide patient data to the application 110 and/or the diabetes management server 104. The patient data may be collected via a third-party application operating on the client device 102 and/or the sensor device 108. For example, a third-party application may collect heartbeat and/or step data, which is transmitted to the third-party server 130 from the sensor device 108 for aggregation and analysis. The third-party server 130 in turn transmits the aggregated and/or analyzed patient data to the application 110 and/or the diabetes management server 104 in relation to specific patient accounts. The third-party server 130 may also acquire patient data through interaction with a web service, such as a meal take out web service, a ride sharing web service, an exercise tracking web service, etc. Additionally, the third-party server 130 may include a medical records provider that stores medical and/or physiological data related to a patient. In some embodiments, the third-party server 130 may also store high-level heath modifications for a diabetes management plan, which may have been created by a patient's medical care provider.

As described in more detail below, the diabetes management server 104 is configured to operate in connection with the application 110 to provide automated lifestyle health coaching for diabetes. In some embodiments, the application 110 is configured as a lite-application for patient data collection, which is transmitted to the management server 104. In these examples, the server 104 includes one or more application programming interfaces (“APIs”). The application 110 is configured to connect to the one or more APIs for transmitting the patient data to the server 104. In some embodiments, certain APIs may be targeted based on the type of patient data to be transmitted.

The management server 104 is communicatively coupled to a memory device 140 having one or more databases. The memory device 140 may include, for example, a MongoDB NoSQL database. In other instances, the memory device 140 may include a SQL database. The memory device 140 is configured to store patient data 142. Each record of patient data 142 is indexed to a particular patient and includes patient data received from the application 110, the user device 102, the clinician device 120, and/or the third-party server 130.

In some embodiments, the memory device 140 may be implemented in a cloud computing environment, such as Amazon® Web Services. The memory device 140 may include SageMaker or other machine learning platform for organizing or otherwise classifying the received patient data. In these instances, the memory device 140 stores the patient data in addition to classifications or summarizations of the patient data.

In the illustrated embodiment, the diabetes management server 104 uses the stored patient data 142 in addition to relevant diabetes clinical guidelines for lifestyle management to determine high-level lifestyle modifications. The diabetes management server 104 then determines actionable recommendations for a patient using their patient data 142 and the high-level lifestyle modifications. The recommendations may include one or more triggers for proxy indicators provided with the patient data 142. The diabetes management server 104 then compares the recommendations to newly received patient data to identify proxy indications for determining if any triggers for the recommendations are to be generated. If a recommendation is to be generated, the diabetes management server 104 determines contextual information for the recommendation using, in part, the newly received patient data 142. The diabetes management server 104 then creates a text message or notification using the contextual information, which is transmitted to the user device 102 and/or the application 110 for display.

In some embodiments, the diabetes management server 104 transmits the actionable recommendations and one or more triggers to the application 110. In these embodiments, the application 110 uses locally acquired patient data and/or any patient data 142 read from the memory device 142 to identify proxy indications for determining if any triggers for the recommendations are to be generated. If a recommendation is to be generated, the application 110 determines contextual information for the recommendation using, in part, the newly received patient data 142. The application 110 then creates a notification using the contextual information, which is displayed on a display screen of the user device 102.

In alternative embodiments, the application 110 accesses the patient data 142 at the memory device 104 via the diabetes management server 104. In these instances, the application 110 determines the high-level lifestyle modifications. Additionally, the application 110 determines a translation of the lifestyle modifications into the actionable recommendations using newly received patient data 142. The application 110 identifies proxy indications in current patient data 142 for determining if any triggers for the recommendations are to be generated. If a recommendation is to be generated, the application 110 determines contextual information for the recommendation using, in part, the newly received patient data 142. The application 110 then creates a notification using the contextual information, which is displayed on a display screen of the user device 102.

As discussed above, at least one proxy indication trigger is associated with an actionable recommendation. In instances where two triggers are associated with an actionable recommendation, the recommendation may be configured to trigger if at least one of the triggers is satisfied or only if both of the triggers are satisfied. For example, an actionable recommendation may correspond to eating a snack at 11:30 AM on Tuesday morning given a number of meetings scheduled on a patient's calendar. The actionable recommendation may include a first proxy indication trigger of Tuesday, a second proxy indication trigger of 11:30 AM, and a third proxy indication trigger corresponding to events being scheduled on the patient's calendar between noon and 2:00 PM. In some instances, the first and second proxy indication triggers are set to being required, while the third proxy indication is set to optional. In these instances, the application 110 and/or the diabetes management server 104 generates the recommendation about consuming a snack as long as proxy indications in current patient data satisfy at least the first and second triggers. Alternatively, in some instances, the first, second, and proxy indication triggers are set to being required. In these instances, the application 110 and/or the diabetes management server 104 generates the recommendation about consuming a snack as long as proxy indications in current patient data satisfy all three of the triggers.

### User Device and Application Embodiment

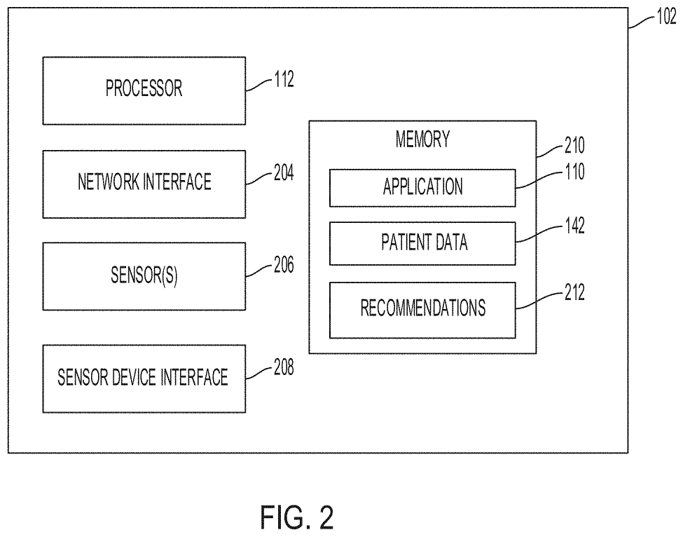

FIG. 2 shows an example diagram of the user device 102, according to an example embodiment of the present disclosure. The user device 102 includes the processor 112, a network interface 204, one or more sensors 206, a sensor device interface 208, and a memory 210. The processor 112 may include a microcontroller, a controller, an application specific integrated circuit (“ASIC”), a central processing unit included on one or more integrated circuits, etc. The memory 210 may include any volatile or non-volatile data/instruction storage device. The memory 210 may include, for example, flash memory, random-access memory (“RAM”), read-only memory (“ROM”), Electrically Erasable Programmable Read-Only Memory (“EEPROM”), etc. The example memory 210 is configured to store one or more instructions defining the application 110, which are executable by the processor 112 to cause the processor 112 to perform operations disclosed herein. The application 110 is configured to request or otherwise receive patient data 142 from the user device 102, the sensor device 108, the third-party server 130, and/or the diabetes management server 104.

In some examples, the application 110 is configured to use the network interface 204 for connecting to one or more interfaces (e.g., APIs) at the diabetes management server 104 for transmitting collected patient data 142. The network interface 204 may include a transceiver and/or port for transmitting and receiving data via the Internet, an Ethernet, a cellular network, etc. In some instances, the application 110 may transmit the patient data 142 in data streams as the data is collected/received. In other instances, the application 110 may transmit the patient data 142 at periodic intervals. In yet other instances, the application 110 may be configured to transmit the patient data 142 at designated times, such as at the end of a day.

The example user device 102 includes one or more sensors 206 for measuring at least some types of patient data. For instance, the user device 102 may include a GPS sensor 206 for determining a latitude and longitude of the user device 102. The user device 102 may also include a six-degree of freedom force sensor 206 to detect linear and angular acceleration. The user device 102 may further include a temperature sensor, a moisture sensor etc. It should be appreciated that the user device 102 and/or the sensor device 108 may include virtually any sensor to measure a parameter or characteristic related to a patient for generating patient data 142.

The application 110 may communicate with registers in the memory 210 and/or processing routines operating on the processor 112 of the user device 102 that store at least a portion of data that can be used as patient data 142. For example, the application 110 may obtain acceleration data from registers configured to store data from a 6 degree-of-freedom sensor and obtain GPS data from a register configured to store GPS data. The application 110 may also communicate with the sensor device interface 208 to receive the corresponding patient data 142 from the sensor device 108. The sensor device interface 208 may include a transceiver for communicatively coupling with the sensor device 108. The senor device interface 208 may include, for example, a Bluetooth® interface, an RF interface, an NFC interface, a USB® interface, or a Lightning® interface.

In addition to obtaining sensor data, the example application 110 is configured to acquire, as patient data 142, device application data of the user device 102. The device application data may include, for example, an application name/type used by a patient, a usage duration, an indication of direct communication, an indication of remote communication, an indication of a photo or video recording, a media type, a sound setting, or a calendar event information. In some instances, the application 110 is configured to operate in a background of the user device 102 to record how a patient uses the device 102. In other examples, the application 110 accesses a task manager to obtain information about application, process, or service usage. Regarding communication monitoring, the application 110 may poll a microphone to detect instances a patient directly communicates with others or communicates via a phone or text messaging application (without making any recording of the patient).

In some embodiments, the application 110 may communicate with third-party applications on the user device 102. For example, the application 110 may communicate with a mapping application to determine location information that corresponds to GPS coordinates. The application 110 may supplement location information with dead-reckoning information from a six degree-of-freedom sensor to estimate a patient location when a GPS signal is not available, such as when a patient travels indoors. In another example, the application 110 may communicate with a health monitor application on the user device 102 to obtain raw and/or calculated health information provided by the health monitor application. In this manner, the application 110 is configured to take advantage of the presence of third-party health monitors or tracking applications to provide a more comprehensive set of patient data 142. For example, the user device 102 may include a step counter application that interfaces with a six degree-of-freedom sensor and/or GPS sensor to estimate a number of steps and a pacing of a patient. Instead of collecting this information separately, the application 110 may be configured to interface with the third-party health monitoring application and/or the third-party server 130 for collecting the patient data 142 for storage in the memory 210.

In addition to the automatic collection of patient data, the example application 110 may include one or more user interfaces configured to enable a patient to enter or self-report certain patient data 142 via text entry or voice entry (which is converted to text by the application 110). The application 110 is configured to store the information entered by the patient into the memory 210 as the patient data 142. In some examples, the application 110 is configured to cause the user interfaces to be displayed at designated times to prompt a patient for patient data 142. In other instances, the application 110 may cause a notification to be displayed on the user device 102, selection of which causes one or more of the user interfaces to be displayed for manual entry of the patient data 142.

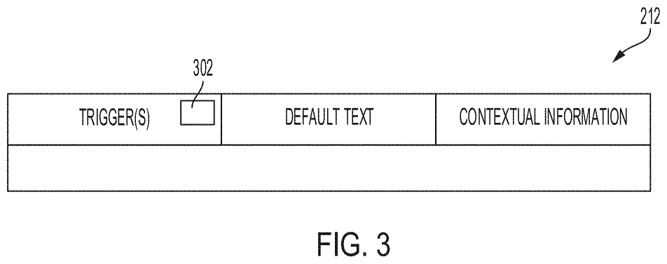

The example memory 210 of the user device 102 may also store actionable recommendations 212 in conjunction with the application 110. The actionable recommendations 212 include a data structure of potential recommendations provided to a patient contingent upon one or more proxy indicator triggers being satisfied. FIG. 3 shows a diagram of an actionable recommendation 212, according to an example embodiment of the present application. The actionable recommendation 212 may be generated from at least one high-level lifestyle modification (e.g., related to exercise or stress) determined by the application 110 and/or the diabetes management server 104. The actionable recommendation 212 includes one or more proxy indication trigger(s) that specify when the recommendation is to be generated for a patient. The proxy indication triggers define Boolean and/or logical conditions based on proxy indications that are included in patient data. The triggers may include GPS coordinates or a location name for a geographic trigger. The triggers may include a day/time for a temporal trigger. The triggers may further include physiological thresholds, such as a glucose level, a heart rate, a blood pressure, a weight, a respiratory rate, activity level, or stress level. The triggers may also include elevation, a patient being inside or outside, season of the year, etc. Further, the triggers may include calendar events, inactivity detection, nutritional intake information, etc.

At least some triggers may include a flag 302. Selection of the flag 302 indicates that the trigger is required to be satisfied in order for the actionable recommendation 212 to be generated by the application 110 and/or the diabetes management server 104. This enables different combinations of triggers to be used for certain actionable recommendations.

As shown in FIG. 3, the actionable recommendation 212 includes at least some default text. The default text provides text for inclusion in a recommendation message. The default text may include common phrases such as “remember to” or “it is recommended to”. The default text may also include fields that reference where contextual information is to be inserted. In some instances, the actionable recommendation 212 may include only default text without contextual information. In these instances, the default text may provide a recommendation, such as “remember to have a snack prior to meetings over lunch”.

As shown in FIG. 3, the actionable recommendation 212 includes a contextual information field that specifies contextual information that is to be included in the message for the recommendation. For example, for a recommendation to avoid a certain location, the contextual information may include an instruction to use a map application to determine a location from coordinates. A name of the location is determined, such as a restaurant name, and then entered into the message. In another example for a food recommendation, the contextual information may include a link to a restaurant menu with programmed logic to identify low sodium and/or low carbohydrate menu items for inclusion in the recommendation message. In yet a further embodiment for activity monitoring, the contextual information may include contents from an activity plan, such as a balance or aerobic exercise.

To determine if an actionable recommendation 212 is to be generated into a recommendation notification message, the application 110 and/or the diabetes management server 104 identifies proxy indications in newly received patient data. As described above, the proxy indications correspond to the conditions of the triggers. The application 110 and/or the diabetes management server 104 determines if the trigger(s) is satisfied by comparing the identified proxy indication to the conditions of the trigger(s). If the trigger(s) is satisfied, the application 110 and/or the diabetes management server 104 generates the recommendation notification message using the default text and contextual information of the corresponding actionable recommendation 212. If the triggers are not satisfied, the application 110 and/or the diabetes management server 104 refrains from generating a recommendation.

It should be appreciated that some actionable recommendations may provide positive feedback. For example, if certain positive triggers are satisfied, such as exercising a certain number of days a week, or eating certain food, the application 110 and/or the diabetes management server 104 may generate a message with the positive feedback in a similar manner as described above. Thus, the application 110 and/or the diabetes management server 104 provides appropriate encouragement and positive feedback to help patients improve their diabetic health.

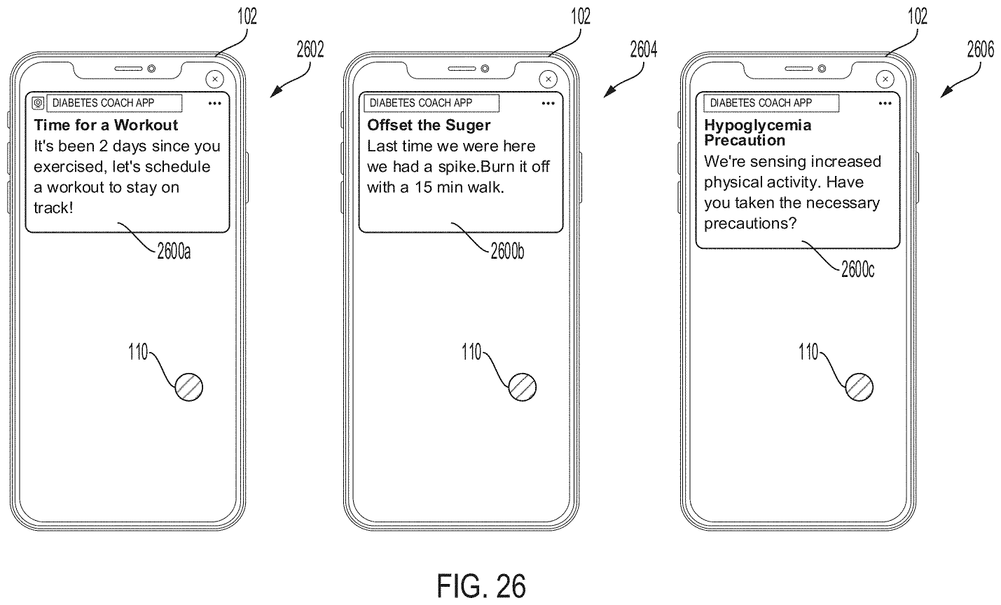

In an example, if the application 110 and/or the diabetes management server 104 has not detected that the patient has performed a workout in a certain number of days, this may trigger the example recommendation notification message 2600a, based on an actionable recommendation 212, that encourages the patient to schedule, or plan, a workout, as shown displayed on the user interface 2602 of the user device 102 in FIG. 26. In another example, if the application 110 and/or the diabetes management server 104 detects that the patient has arrived at a location where the patient typically experiences a blood sugar spike, this may trigger the example recommendation notification message 2600b, based on an actionable recommendation 212, that encourages the patient to perform exercise to burn off a likely blood sugar spike, as shown displayed on the user interface 2604 of the user device 102 in FIG. 26.

In another example, if the application 110 and/or the diabetes management server 104 detects that the patient has started to increase physical activity (e.g., started a workout), this may trigger the example recommendation notification message 2600c, based on an actionable recommendation 212, that queries whether the patient has taken the necessary precautions before engaging in a workout, as shown displayed on the user interface 2606 of the user device 102 in FIG. 26. The recommendation notification message 2600c can serve as an aid to help prevent the patient from experiencing a hypoglycemic episode.

### Patient Data and Lifestyle Activity Detection Embodiments

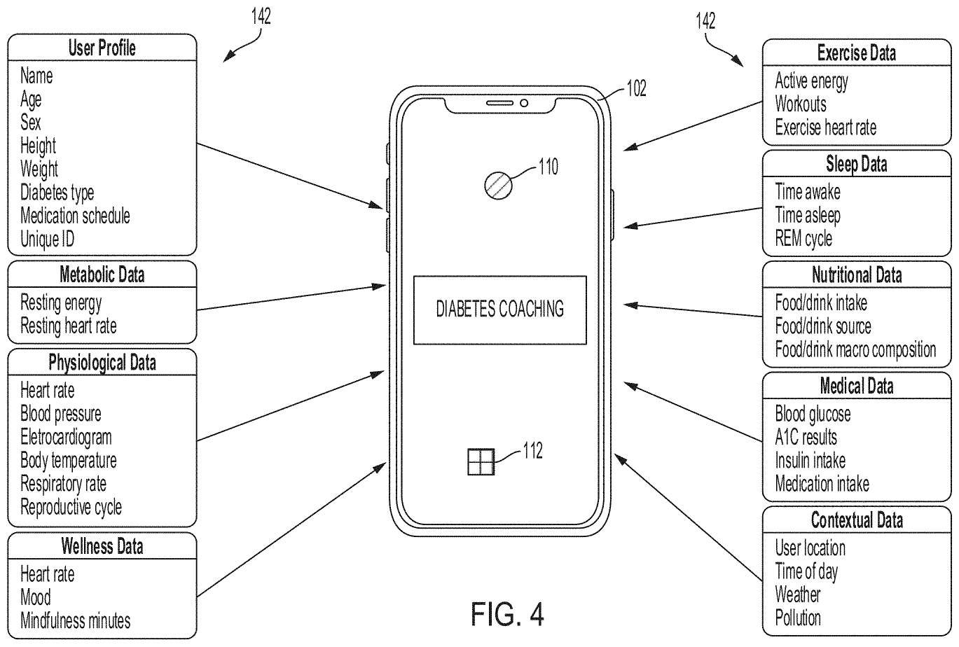

In various embodiments, the application 110 and/or the diabetes management server 104 may receive holistic, time-correlated data about a user's context, environment, behavior, and physiology. FIG. 4 shows a diagram of example types of patient data 142 that may be received by the application 110 and/or the diabetes management server 104, according to an example embodiment of the present disclosure. The types of patient data 142 shown in FIG. 4 are only illustrative. In other embodiments, fewer or more types of patient data may be used. The types of patient data 142 shown may be used for creating actionable recommendations 212 including proxy triggers. In other instances, the types of patient data 142 may be used for identifying proxy indications for comparison to triggers. Further, the types of patient data 142 may provide contextual information for inclusion in an actionable recommendation 212.

As shown in FIG. 4, the patient data 142 may be obtained from one or more sources including the user device 102, the sensor device 108, the application 110, the diabetes management server 104, and/or the third-party application server 130. The patient data 142 may be directly input or measured. Alternatively, the patient data 142 may be derived from rules-based models and/or machine learning models of a patient's lifestyle. The patient data 142 may include any one or more of a patient name, age, sex, height, weight, diabetes type, medication schedule, unique identifier, resting energy, resting heart rate, heart rate, step count, blood pressure, electrocardiogram information, body temperature, respiratory cycle or rate, reproductive cycle, mood, mindfulness minutes, active energy, workout information, exercise heart rate, time awake, time asleep, REM cycle information, food/drink intake, a food/drink source, a food/drink macro composition, blood glucose measurement, A1C results, insulin intake, medication intake, a patient location, a time of day, the weather in proximity to the patient, and/or reported pollution levels in proximity to the patient. Other example patient data may include stress, season of the year or number of people in the patient's location, patient interactions with the application 110, patient's susceptibility to behavior change, patient preferences (e.g., foods, drinks, or activities, etc.), patient reactions to various stimuli (e.g., does seeing a bad blood sugar result cause the user to avoid taking future measurements), patient lifestyle patterns, frequented restaurants, etc.

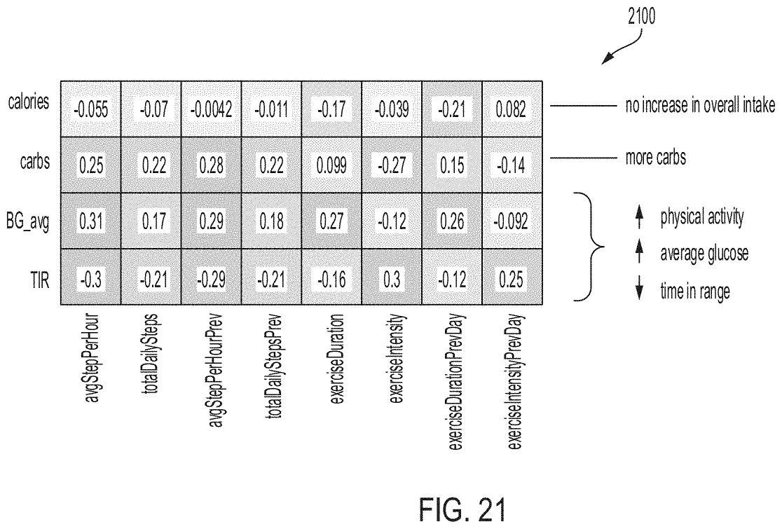

The time-correlated nature of this patient data 142 received by the application 110 and/or the diabetes management server 104 enables the identification of correlations among these factors. For example, monitoring glucose data correlated with location data can enable detecting nutritional intake events. In another example, certain locations may be correlated to hypoglycemic events, and the user can be alerted upon arrival at such a location to take precautions. In another example, carbohydrate intake, metabolic energy expenditure and blood glucose can be correlated to determine a physiological model for the user to process glucose and insulin. In another example, correlating weather data with nutritional intake and blood glucose readings can create a model of how weather changes may impact a user's eating habits and therefor affect the user's management of diabetes. FIG. 21 illustrates an example holistic model 2100 that correlates exercise duration and intensity with calories consumed, carbohydrates consumed, average blood glucose (BG_avg), and an amount of time the patient's blood glucose is within upper and lower targets of a desired range (“TIR”). As such, the holistic model 2100 demonstrates the importance of exercise as part of a holistic patient model for predicting how different patient parameters change. Stress can similarly play an important role as part of a holistic patient model for predicting how different patient parameters change.

As stated above, the application 110 and/or the diabetes management server 104 may detect various lifestyle activities of a patient as triggers for generating actionable recommendations 212. The application 110 and/or the diabetes management server 104 detects these lifestyle activities based on received patient data 142, which may include physiological data, such as patient heart rate data. For example, exercise can typically be identified by elevated heart rates. A patient may wear a fitness tracking device, such as a smartwatch or heart rate monitor, that monitors (e.g., via the sensor device 108) the patient's heart rate and transmits that data to the application 110 and/or the diabetes management server 104.

Typical fitness tracking devices, however, will record a user's heart rate every 5 to 10 minutes to conserve battery life, since a user's heart rate does not vary significantly during most of the day, unless a “record” setting is activated that increases the heart rate sample rate to about every five seconds. As such, if a user activates the “record” setting at the start of the user's workout and deactivates it at the end, then the typical fitness tracking device will generally record the start and end of the user's workout based on the differences in sample rate of the data collected. If, however, the user forgets to activate the “record” setting when beginning to work out, the typical fitness tracking device will continue to collect data at a sample rate of 5 to 10 minute intervals, omitting significant exercise data. For instance, a 15-minute workout routine may only have a single data point with the elevated heart rate of the patient. In many instances, a single data point for an increased heart rate may not be sufficient to classify an activity as exercise. Accordingly, data received from a typical fitness tracking device is not always sufficient alone to detect that a patient completed a workout or to detect a start and end of a workout.

In various embodiments, the application 110 and/or the diabetes management server 104 may detect that a patient completed a workout despite receiving infrequent and/or unreliable (e.g., high stress rather than exercise) heart rate data. For instance, in such embodiments, the application 110 and/or the diabetes management server 104 enables detecting that a patient completed a workout despite the patient forgetting to activate the “record” setting on the patient's fitness tracking device. To do so, the application 110 and/or the diabetes management server 104 may utilize data indicative of the patient completing a workout other than heart rate data. For example, step count data may be received for a patient at a higher frequency than the heart rate data and therefore a step count rate determined from that step count data may be a better primary indicator that the patient started to work out than the infrequent heart rate data. For instance, the patient might work out for 5 to 10 minutes prior to a first heart rate data point being received, but the patient's step count rate might be elevated during that 5 to 10 minutes thereby indicating the patient is working out.

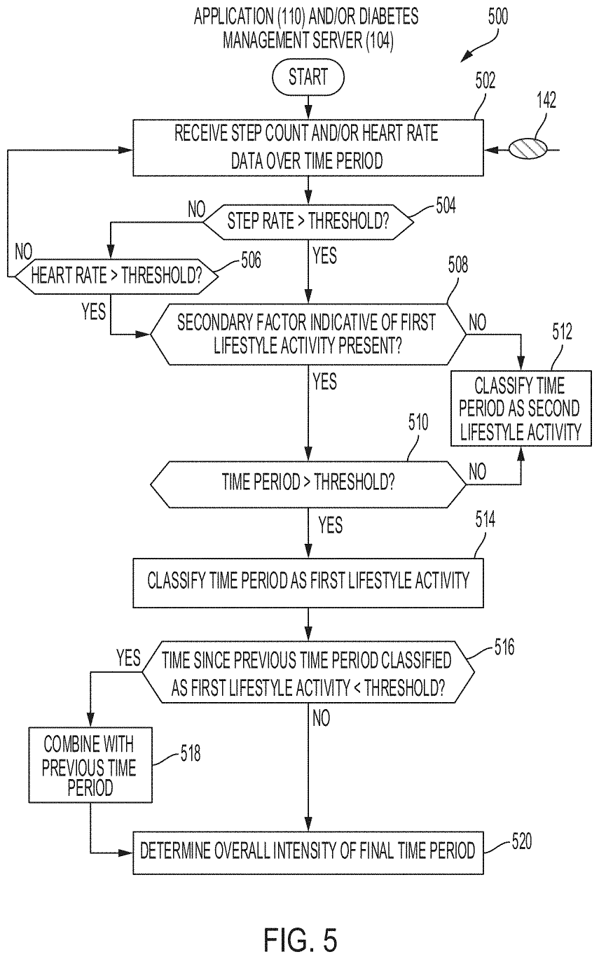

FIG. 5 illustrates a flow diagram of an example procedure 500 for detecting lifestyle activities, according to an example embodiment of the present disclosure. Although the procedure 500 is described with reference to the flow diagram illustrated in FIG. 5, it should be appreciated that many other methods of performing the steps associated with the procedure 500 may be used. For example, the order of many of the blocks may be changed, certain blocks may be combined with other blocks, and many of the blocks described may be optional. In an embodiment, the number of blocks may be changed. The actions described in the procedure 500 are specified by one or more instruction and may be performed among multiple devices including, for example, the application 110 on the user device 102 and/or the diabetes management server 104.

The example procedure 500 begins when patient data 142 including step count data and/or heart rate data is received in the application 110 and/or the diabetes management server 104 (block 502). The step count data and/or heart rate data may be received over a time period. The application 110 and/or the diabetes management server 104 then determines whether a rate of steps (e.g., steps/minute) in the step count data over the time period is greater than a predefined step rate threshold (block 504). In various aspects, the application 110 and/or the diabetes management server 104 may determine if an accumulation of the step data over minute intervals exceeds the predefined step rate threshold. In some aspects, the predefined step rate threshold is defined to be a step rate indicative of at least a light workout. For example, the predefined step rate threshold may be at least 20 steps per minute and as many as 40 to 75 steps per minute.

In some aspects, an activity (e.g., walking/running, cycling, rollerblading, etc.) that a patient is performing can be detected and the predefined step rate threshold may be set based on that detected activity. For example, the application 110 and/or the diabetes management server 104 may detect patterns (e.g., a frequency between steps) from data corresponding to a particular activity in order to detect that particular activity in the future. In such aspects, a lower step rate threshold (e.g., 30 steps per minute) may be used for cycling as compared to running. It will be appreciated that different step rates will correspond to a light workout for different patients. As such, the predefined step rate threshold may be set based on data particular to a specific patient, such as data related to distances traveled, movement speeds, accelerometer data patterns, and the like.

If it is determined that the patient step count rate is below the predefined step rate threshold, the application 110 and/or the diabetes management server 104 then determines whether at least one heart rate data point over the time period is greater than a predefined heart rate threshold (block 506). In an example, a patient may perform a workout that includes a low rate of steps, or at least a rate of steps below the predefined step rate threshold, such as a resistance, or weight lifting, workout. In such an example, the patient's heart rate data may be the best primary indicator that the patient started to work out, or is continuing to work out (e.g., after a warm up that included a high step rate), regardless of the frequency of that heart rate data. In at least some aspects, the predefined heart rate threshold is defined to be a heart rate indicative of at least a light workout. For example, in some aspects, the predefined heart rate threshold may be based on a patient's maximum heart rate. In such aspects, a light workout may correspond to 57-63% of the patient's maximum heart rate. In one example, a patient's maximum heart rate can be calculated by the formula HRₘₐₓ=208−(0.7×age). In other examples, a patient's maximum heart rate can be determined using other suitable methods. In other aspects, the predefined heart rate threshold may be based on a heart rate set by the patient or a clinician, based on clinical guidance or demographically based values, based on feedback from algorithm training, or other suitable methods for setting a heart rate threshold indicative of at least a light workout.

If it is determined that the patient's heart rate data does not include a heart rate above the predefined heart rate threshold, then the application 110 and/or the diabetes management server 104 continues to receive patient data 142 at block 502. In some aspects, the application 110 and/or the diabetes management server 104 may also classify the time period as the second lifestyle activity at this point, the second lifestyle activity being described more below. Conversely, if it is determined that the patient step count rate is greater than the predefined step rate threshold at block 504 or the patient's heart rate data includes a heart rate above the predefined heart rate threshold at block 506, then the application 110 and/or the diabetes management server 104 determines whether a secondary factor indicative of a first lifestyle activity (e.g., a workout) is present in the patient data 142 (block 508). In some embodiments, the application 110 and/or the diabetes management server 104 may determine whether more than one secondary factor indicative of the first lifestyle activity is present in the patient data 142.

Determining whether a secondary factor indicative of a workout is present can help reduce an error rate of the application 110 and/or the diabetes management server 104 in determining whether the patient performed a workout. For example, the application 110 and/or the diabetes management server 104 may receive step data indicating the patient has a step rate exceeding the predefined step rate threshold for a time period, though the patient is actually just walking to work rather than working out. In this example, a secondary factor could be the patient's heart rate exceeding the predefined heart rate threshold in combination with the step rate being the primary indicator. For instance, the patient's heart rate might not exceed the predefined heart rate threshold on the patient's walk to work therefore helping the application 110 and/or the diabetes management server 104 distinguish between the patient's commute and a workout. In another example, a secondary factor could be the patient's location determined from location information received for the patient, in combination with the step rate being the primary indicator. For instance, it is more likely that the patient is working out if the patient's step rate exceeds the threshold and the patient's location is at the gym rather than on a street near the patient's office. In another example, a secondary factor could be a rate of location change for the patient determined from location information received for the patient, in combination with the step rate being the primary indicator. For instance, a higher rate of location change with a step rate exceeding the threshold can indicate that the patient is running rather than walking, thus making it more likely that the patient is working out.

Additionally, the application 110 and/or the diabetes management server 104 may receive heart rate data indicating that the patient has a heart rate exceeding the predefined heart rate threshold, though the patient might not be working out. For instance, the patient's elevated heart rate could be due to stress or another inducive factor. As such, the patient's location or patient's rate of location change can be secondary factors, in combination with the heart rate being the primary indicator, that make it more likely that the patient is working out. In another example, while a patient's step count rate might not exceed the predefined step rate threshold, a step rate greater than zero can be a secondary factor in combination with the heart rate being the primary indicator. For instance, a step rate greater than zero can indicate that the user is moving in combination with an elevated heart rate, rather than being stationary, thus making it more likely that the patient is working out. In some aspects, a secondary factor could be a step rate meeting or exceeding a threshold that is greater than zero but less than (e.g., 50%, 75%, etc.) the predefined step rate threshold. For instance, a patient with an elevated heart rate due to stress might take some steps and have a step rate greater than zero, but does not take as many steps as a patient performing a weight training workout, and therefore including the threshold can, in some instances, help generate a more accurate determination that the patient is working out.

If it is determined that a secondary factor indicative of the first lifestyle activity is present in the patient data 142, then the application 110 and/or the diabetes management server 104 determines whether a length of the time period is greater than a predefined time threshold (block 510). The predefined time threshold may be set such that it is indicative of at least a short burst of activity (e.g., 1 minute) that may be performed in a workout, such as interval training, though the predefined time threshold may alternatively be indicative of a longer stretch of activity (e.g., 5-10 minutes). Determining whether the length of the time period is greater than the predefined threshold can help reduce an error rate of the application 110 and/or the diabetes management server 104 in determining whether the patient performed a workout. For instance, the application 110 and/or the diabetes management server 104 would not determine that a quick 15-30 second run to catch a bus was a workout since it did not meet the predefined time threshold.

In one aspect, the predefined time threshold may be combined with a secondary factor in order to determine whether the predefined time threshold is satisfied. For example, a time period of 1 minute might only satisfy the predefined time threshold if it is within a certain amount of time from another time period that satisfies blocks 504-508. This is similar to combining time periods as described below. Conversely, a time period of 5-10 minutes or more might not have that secondary factor requirement because a time period of 5-10 minutes or more is more likely to be part of a workout. Such an example can help distinguish between an isolated short burst of activity and a short burst of activity that is part of an interval training workout.

Additionally, determining whether a length of the time period is greater than a predefined time threshold includes determining the start and end of the time period in order to determine the length of the time period. In some aspects, the application 110 and/or the diabetes management server 104 determines the start and end of the time period based on the received step data. For instance, the start of a workout will typically have a higher rate of steps than the time period just prior to the start of the workout, and the period directly after a workout will often times have a lower rate of steps than the workout itself. In such aspects, the time point at which it is determined that the rate of steps in the step count data is greater than the predefined step rate threshold may be defined as the start of the time period. Similarly, a subsequent time point at which it is determined that the rate of steps is less than or equal to the predefined step rate threshold may be defined as the end of the time period.

In other aspects, the application 110 and/or the diabetes management server 104 determines the start and end of the time period based on the received heart rate data. In such aspects, the time point at which it is determined that the heart rate data includes heart rate(s) above the predefined heart rate threshold may be defined as the start of the time period. Similarly, a subsequent time point at which it is determined that the patient's heart rate is less than or equal to the predefined heart rate threshold may be defined as the end of the time period. It should be appreciated, however, that the example process 500 is an iterative process. For example, the application 110 and/or the diabetes management server 104 may determine that the step rate exceeds the step rate threshold combined with a secondary factor that the patient's heart rate exceeds the heart rate threshold, which thereby indicates a start of the time period. The application 110 and/or the diabetes management server 104 then determining that the next subsequent step data point after the start of the time period is equal to or below the step rate threshold does not necessarily signify the end of the time period. For instance, at the time of that next subsequent step data point, the application 110 and/or the diabetes management server 104 may determine that the patient's heart rate is above the heart rate threshold combined with a secondary factor indicative of the first lifestyle activity (e.g., the patient's location is at the gym) despite the step count rate being below the step rate threshold, which may thereby indicate that the time period has not ended.

In some instances, the step data might not pinpoint the start and end of the workout, or the application 110 and/or the diabetes management server 104 might not receive step data. Additionally, as discussed, the heart rate data may be received at a low sampling rate therefore limiting the accuracy with which the heart rate data points can determine the true start and end of the time period. For example, if heart rate values are used as a trigger for detecting the start and end of a workout, there is a period between the last non-workout heart rate sample and the first workout heart rate sample during which the patient has started working out. Similarly, there is a period between the last workout heart rate sample and the first non-workout heart rate sample during which a patient may have continued to exercise before stopping the workout. In such instances, the application 110 and/or the diabetes management server 104 may employ other methods to help increase the accuracy with which the start and end of the time period are determined.

In one such method, the application 110 and/or the diabetes management server 104 may perform an interpolation between heart rate data values. For instance, the application 110 and/or the diabetes management server 104 may perform an interpolation between the last non-workout heart rate sample and the first workout heart rate sample to more accurately determine a true start of the workout. The application 110 and/or the diabetes management server 104 may also perform an interpolation between the last workout heart rate sample and the first non-workout heart rate sample to more accurately determine the true end time point of the workout. Suitable interpolation techniques that the application 110 and/or the diabetes management server 104 may implement include linear or exponential interpolation between two points.

Another such method is the application 110 and/or the diabetes management server 104 may add or substitute a warm-up or cool-down curve to the beginning or end of the data, respectively, based on at least one of a patient's historical data, typical data for a particular type of workout, a function of the total duration of the time period, a function of the time duration between two points (e.g., the last non-workout heart rate and the first workout heart rate), a function of the difference between the workout and non-workout data (e.g. change in heart rate), or other suitable models. For example, if a patient typically performs a five minute warm-up routine prior to every workout, the application 110 and/or the diabetes management server 104 may generate a warm-up curve based on heart rate data points received during at least one, and potentially many, of the patient's warm up routines. The application 110 and/or the diabetes management server 104 can then add this generated warm-up curve to the beginning of the heart rate data received for the time period or substitute this generated warm-up curve for a portion of the beginning of the heart rate data received for the time period. In some aspects, the application 110 and/or the diabetes management server 104 may add the generated warm-up curve and interpolate between the last data point of the warm-up curve and the first heart rate data point received in the time period. It will be appreciated that the cool-down curve can be generated similar to the warm-up curve.

An additional example method for determining the start and end of the time period is the application 110 and/or the diabetes management server 104 may utilize location information of the patient or a rate of location change of the patient. For example, a patient may leave the gym prior to the patient's heart rate decreasing back to a resting baseline and therefore the patient's heart rate data may indicate that the patient is still working out after the patient has left the gym. The patient's location, however, can be used to determine that the patient is no longer at the gym and therefore that the workout was completed prior to the location change.

In another example, a patient may be working out on a cardio machine (e.g., treadmill, stationary bike, elliptical, etc.) and therefore the patient's location does not change or changes minimally during the workout. In such an example, the cessation of location change for the patient could indicate when the patient precisely started to use the cardio machine, and when the patient again changes location could indicate when the patient stopped using the cardio machine. In another example, a patient may be running outside and have a faster rate of location change during the run as compared to just prior to, and just after, the run which can be used to indicate the start and end times of the run.

A further example method for determining the start and end of the time period is the application 110 and/or the diabetes management server 104 may query a patient for precise or estimated start and end times. For instance, the patient may use the user device 102 to input that they have started a workout at the time they start the workout, and to input that they have completed the workout at the time they complete the workout. Alternatively, the patient may input start and stop times at some point after completing the workout.

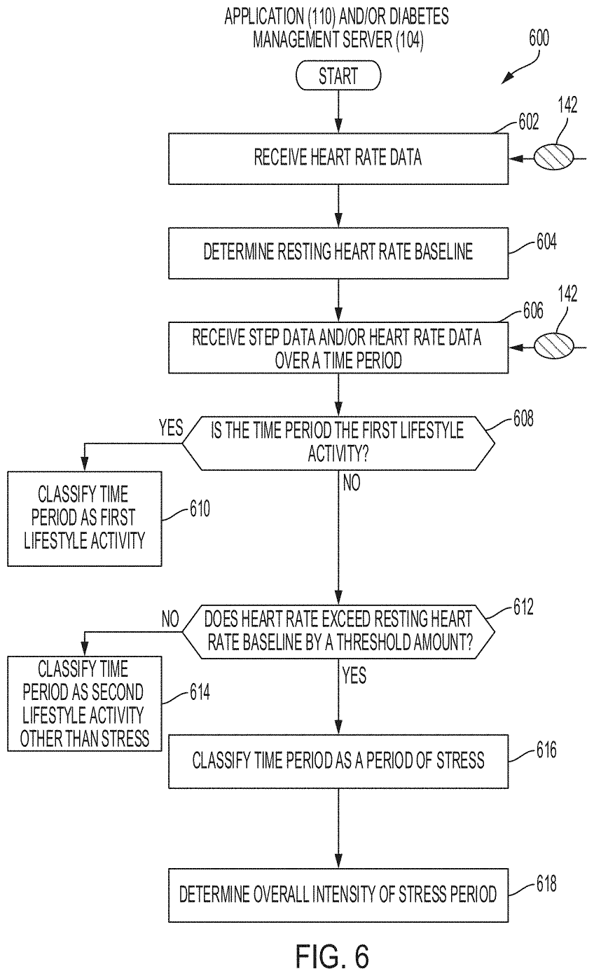

If it is determined that a secondary factor indicative of the first lifestyle activity is not present in the patient data 142 or that the length of the time period does not meet the predefined time threshold, then the application 110 and/or the diabetes management server 104 classifies the time period as a second lifestyle activity (block 512). The second lifestyle activity may be an activity other than working out. In some instances, a second lifestyle activity may involve steps or cause an elevated heart rate, though this is not always the case. For instance, the second lifestyle activity could be watching television, reading a book, playing video games, commuting to work and/or working, a leisurely walk, or an activity inducing stress. A procedure for determining whether the second lifestyle activity is an activity inducing stress in a patient is discussed in more detail below (FIG. 6). In some aspects, classifying the time period as a second lifestyle activity might only involve the application 110 and/or the diabetes management server 104 refraining from classifying the time period as the first lifestyle activity and continuing to receive patient data 142.

If it is determined that the length of the time period does meet the predefined time threshold, then the application 110 and/or the diabetes management server 104 classifies the time period as the first lifestyle activity (block 514). The application 110 and/or the diabetes management server 104 then determines whether the time that has elapsed between a previous time period classified as the first lifestyle activity and the time period classified as the first lifestyle activity in block 512 is less than a predefined threshold (e.g., 5 minutes, 10 minutes, 15 minutes, etc.) (block 516). If the elapsed time is less than the predefined threshold, then the application 110 and/or the diabetes management server 104 combines the time period classified as the first lifestyle activity in block 512 with the previous time period (block 518). For instance, a patient may complete a portion of a workout (e.g., the previous time period), take a rest break, and then complete another portion of a workout (e.g., the time period classified at block 512), though both portions are a part of the same workout. Therefore, rather than recording each portion as a separate workout, which may confuse the patient, the application 110 and/or the diabetes management server 104 combines both portions into the same workout having a duration across both portions.

In some aspects, the application 110 and/or the diabetes management server 104 may determine an overall intensity of the final first lifestyle activity time period, whether or not the time period classified at block 512 is combined with a previous time period (block 520). Stated differently, the application 110 and/or the diabetes management server 104 determines an overall intensity of the time period classified at block 512 if the elapsed time since the previous time period classified as the first lifestyle activity is greater than the predefined threshold. Conversely, the application 110 and/or the diabetes management server 104 determines an overall intensity of the combined time period, including the time period classified at block 512 and the previous time period classified as the first lifestyle activity, if the elapsed time since the previous time period is less than the predefined threshold.

In an example, an overall intensity of the final time period may be determined based on how the patient's heart rate data during the final time period compares to the patient's pre-calculated maximum heart rate. More specifically, in this example the intensity categories include a very light intensity that corresponds to less than 57% of the patient's pre-calculated maximum heart rate, a light intensity that corresponds to 57-63%, a moderate intensity that corresponds to 64-76%, a vigorous intensity that corresponds to 77-95%, and a near maximal to maximal intensity that corresponds to greater than or equal to 96%. In various aspects of this example, the application 110 and/or the diabetes management server 104 determines an average, median, and/or maximum heart rate value over the final time period, calculates what percentage that value(s) is of the patient's pre-calculated maximum heart rate, and compares that calculated percentage with the above ranges. If only the average, median, or maximum heart rate is determined, then the single calculated percentage determines the overall intensity. If more than one are determined, then in various aspects the lowest, middle, or highest category among the calculated percentages may be selected as the overall intensity.

In one aspect, the application 110 and/or the diabetes management server 104 may determine the overall intensity of the final time period with the max-count approach. In the max-count approach, the intensity category that includes the most heart rate data points during the final time period is the determined intensity category for the final time period.

Once the intensity category for the final time period is determined, the application 110 and/or the diabetes management server 104 stores the final time period with its intensity category to a daily activity log of the patient as part of a health log and feedback routine. For example, the application 110 and/or the diabetes management server 104 may record that the patient completed a workout of the determined intensity on the day and at the time corresponding to the final time period. The application 110 and/or the diabetes management server 104 may then return to collecting and analyzing patient data 142 for the next time period or event.

In various embodiments, the application 110 and/or the diabetes management server 104 may generate heart rate and/or step distributions (e.g., over hours of the day, days of the week, months of the year, etc.) that may be displayed on a display screen of the user device 102. The displayed heart rate and/or step distributions can enable a time-correlated analysis of the patient's active periods (e.g., workouts) during a day, week, etc. In at least some instances, the displayed heart rate and/or step distributions can enable a patient or clinician to visualize a time period determined to correspond to a first lifestyle activity (e.g., a workout) with the data used to make that determination.

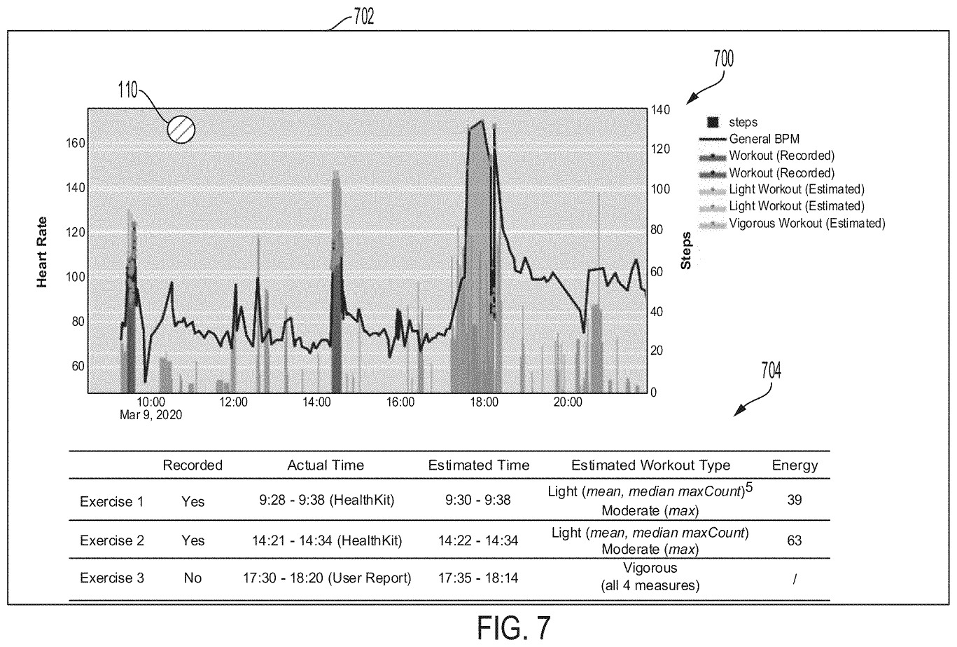

FIG. 7 illustrates a user interface 702 displaying, on a display screen of the user device 102, an example graph 700 showing a distribution of heart rate data and step data for a patient over a portion of the day. The graph 700 may be generated by the application 110 and/or the diabetes management server 104, though only the application 110 is indicated in FIG. 7. In some aspects, the user interface 702 may also display a data table 704 corresponding to the graph 700 on the display screen of the user device 102. In some aspects, an intensity of a workout may be color-coded on the graph 700 so that a patient or clinician may quickly distinguish between the different intensities.

In this example, the patient started the “record” setting on the patient's fitness tracking device for the time periods of Exercise 1 and Exercise 2. The true start and end time of the patient's workout (e.g., Actual Time) for Exercise 1 and Exercise 2 was therefore determined by the higher frequency heart rate data during the period that the “record” setting was started (e.g., HealthKit). The application 110 determined an “Estimated Time” start and end for each of Exercise 1 and Exercise 2 from the received step data. As shown, the start and end times estimated by the step data closely match the actual times determined by the higher frequency heart rate data. For Exercise 3 in this example, the patient failed to start the “record” setting on the patient's fitness tracking device. Rather, the “Actual Time” for Exercise 3 was estimated by the patient in response to a query from the application 110 (e.g., User Report). As shown, the start and end times estimated by the step data are different than the times estimated by the patient. The example procedure 500 can therefore help increase the accuracy with which the application 110 determines a workout duration, since patients may often overestimate the amount of time that they workout.

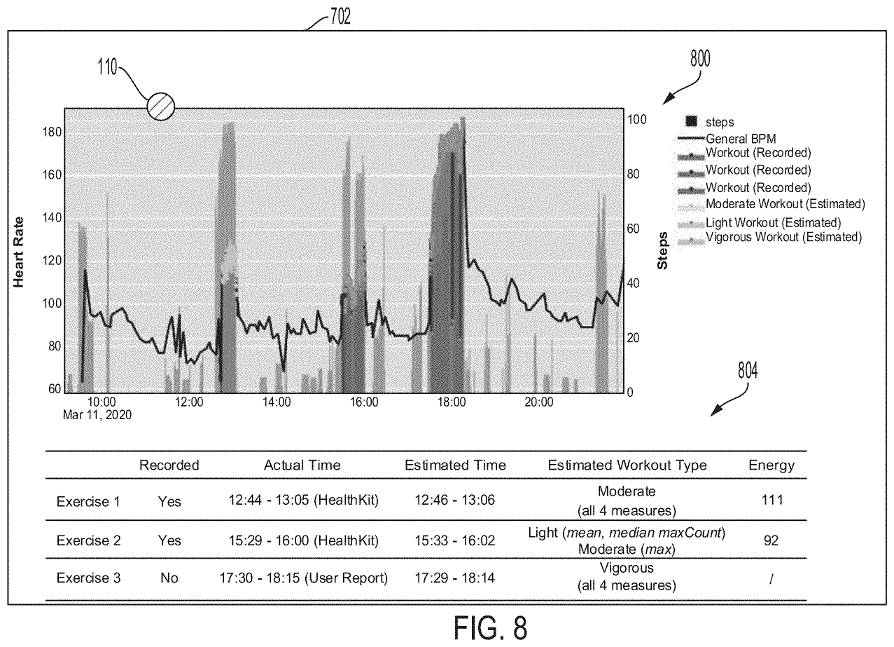

FIG. 8 illustrates the user interface 702 displaying, on a display screen of the user device 102, an example graph 800 showing a distribution of heart rate data and step data for a patient over a portion of the day. The graph 800 may be generated by the application 110 and/or the diabetes management server 104, though only the application 110 is indicated in FIG. 8. In some aspects, the user interface 702 may also display a data table 804 corresponding to the graph 800 on the display screen of the user device 102. In some aspects, an intensity of a workout may be color-coded on the graph 800 so that a patient or clinician may quickly distinguish between the different intensities. The graph 800 and data table 804 provide an additional example in which the patient started the “record” setting on the patient's fitness tracking device for the time periods of Exercise 1 and Exercise 2, but failed to start the “record” setting for the time period of Exercise 3. As shown, the “Actual Time” and “Estimated Time” closely match for Exercise 1 and Exercise 2. The “Actual Time” and “Estimated Time” also closely match for Exercise 3, thus indicating that the patient made an accurate estimate of their workout duration.

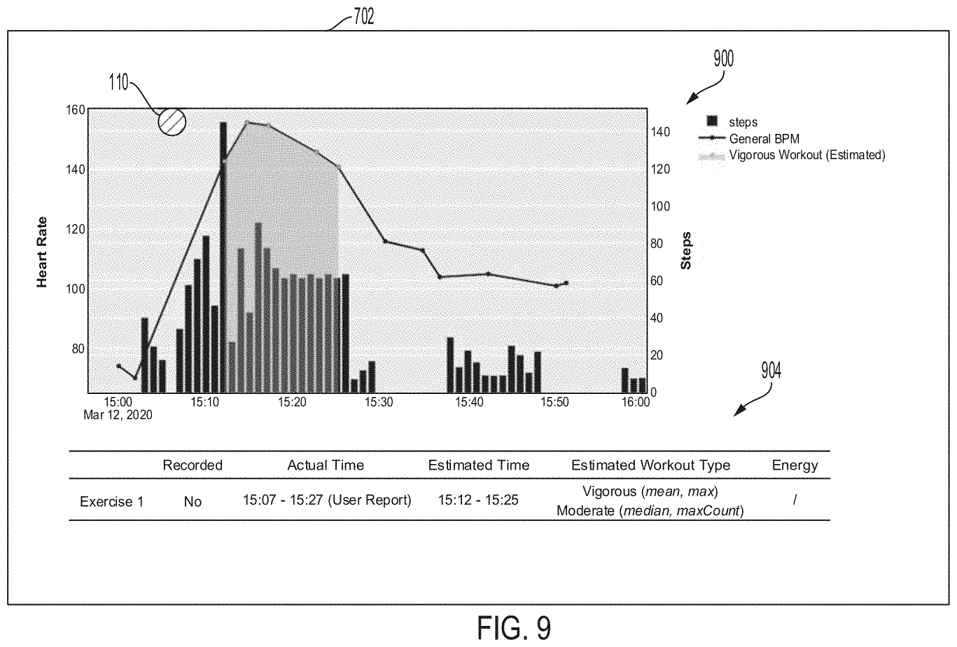

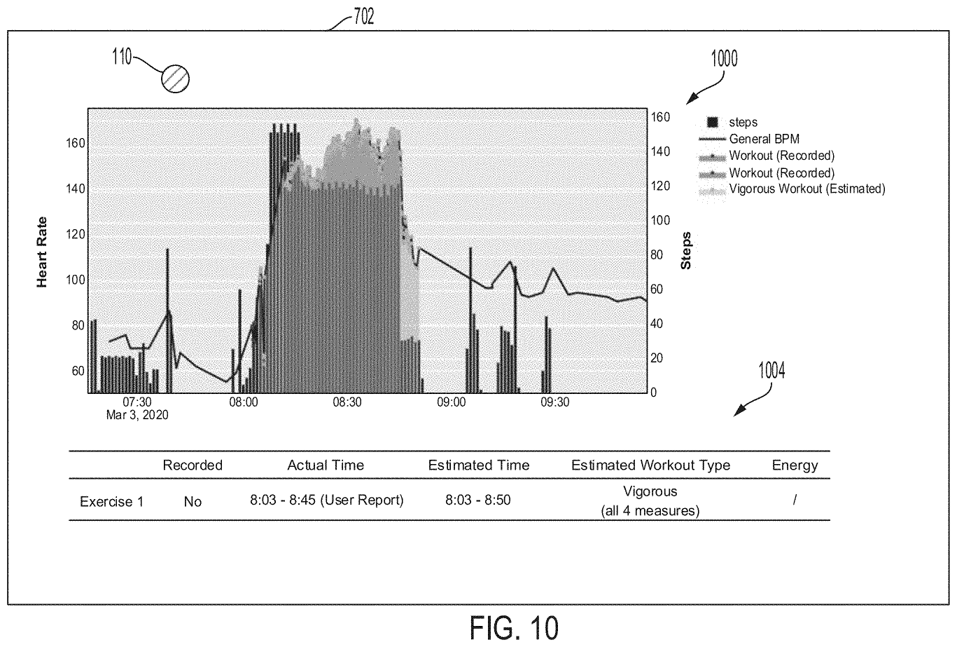

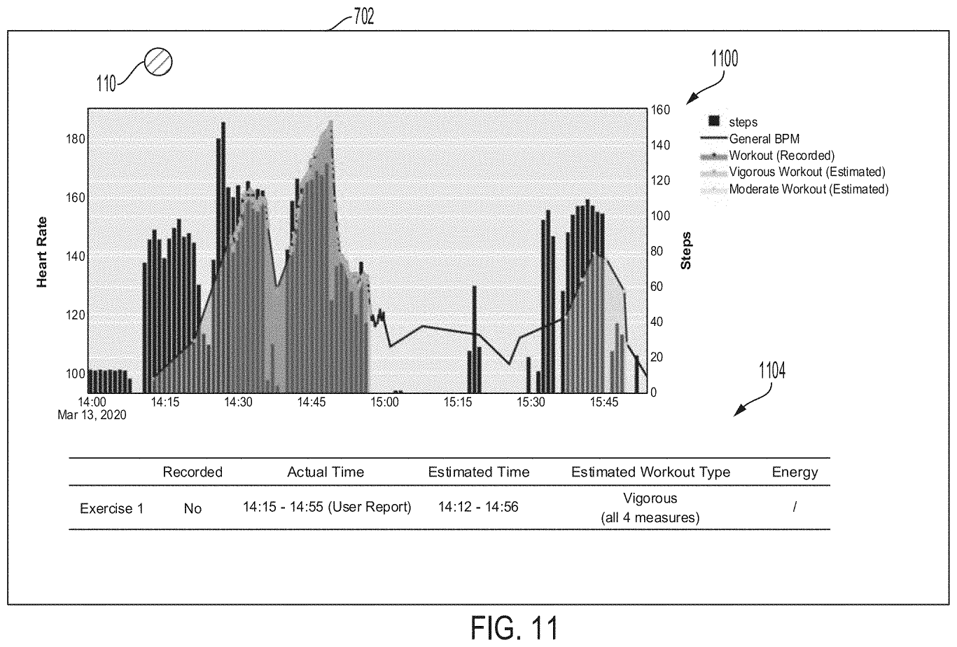

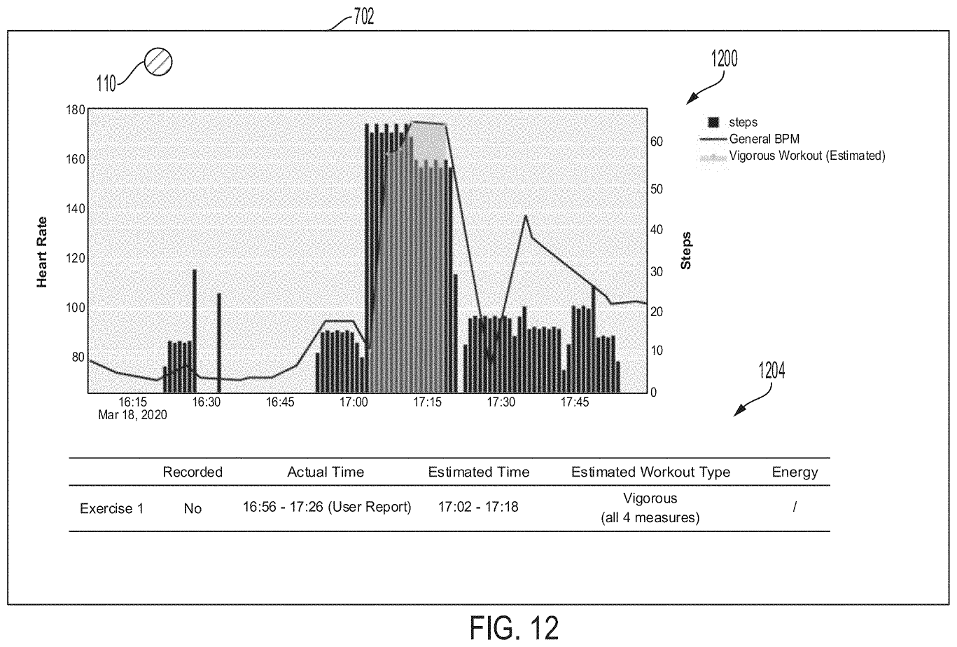

FIGS. 9 to 12 each respectively illustrate the user interface 702 displaying, on a display screen of the user device 102, graphs 900, 1000, 1100, or 1200 showing distributions of heart rate data received over a time period at a low sampling rate and of step data over a time period, from which the application 110 and/or the diabetes management server 104 may determine whether the time period is the first lifestyle activity. FIGS. 9 to 12 also illustrate the data tables 904, 1004, 1104, and 1204 that correspond to the respective graphs 900, 1000, 1100, and 1200. Each of the graphs 900, 1000, 1100, and 1200 may be generated by the application 110 and/or the diabetes management server 104, though only the application 110 is indicated in FIGS. 9 to 12. Further, the user interface 702 may display each of the graphs 900, 1000, 1100, or 1200 on the display screen of the user device 102.

In these examples of FIGS. 9 to 12, the application 110 and/or the diabetes management server 104 determines that the first lifestyle activity (e.g., workout) starts when both the step rate exceeds a step rate threshold that corresponds to a light workout and, as a secondary factor, the heart rate exceeds a heart rate threshold that corresponds to a light workout. Though not shown in FIGS. 9 to 12, as described above, the application 110 and/or the diabetes management server 104 may use interpolation of the heart rate data to identify the start and/or end of the exercise activity.

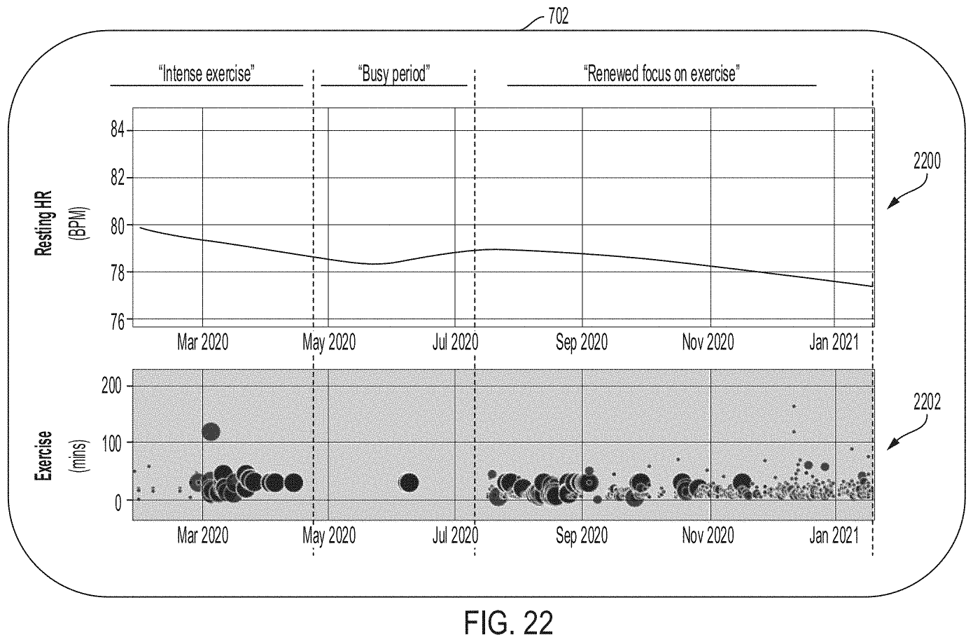

One advantage of detecting periods when the patient has completed a workout is that the application 110 and/or the diabetes management server 104 can correlate the patient's workout data, or lack thereof, with other patient information in order to provide active guidance regarding the patient's cardiovascular fitness or various other aspects of the patient's life. For example, FIG. 22 illustrates the user interface 702 displaying, on a display screen of the user device 102, a graph 2200 that charts the patient's detected resting heart rate from just prior to March 2020 to just after January 2021, and a graph 2202 that charts the patient's detected exercise periods during the same time period. As shown, from March 2020 until about May 2020 the patient performed many periods of exercise, or workouts, and many had a high intensity level designated by the larger circles. During this same time period, the patient's resting heart rate gradually decreased as the patient's cardiovascular fitness improved.

Between about May 2020 and about July 2020, however, the patient largely ceased performing any workouts and the patient's resting heart rate started to gradually increase accordingly. At this point, when the application 110 and/or the diabetes management server 104 does not detect that the patient has been performing workouts, or detects that the frequency with which the patient performs workouts has been decreasing, the application 110 and/or the diabetes management server 104 can generate a recommendation notification message, based on an actionable recommendation 212, that alerts the patient or the patient's clinician to the patient's deteriorating cardiovascular fitness. Generating a recommendation notification message including information of this sort can help course-correct the patient back on a better path of performing workouts in order to improve the patient's diabetic health. For instance, after generating the recommendation notification message alerting the patient or the patient's clinician in July 2020 of the patient's deteriorating cardiovascular fitness, the patient began exercising again and the patient's resting heart rate accordingly began to gradually decrease again. In addition, the application 110 and/or the diabetes management server 104 can, at least in some instances, detect the patient's deteriorating cardiovascular fitness earlier than the patient's clinician and can therefore help course-correct the patient sooner.

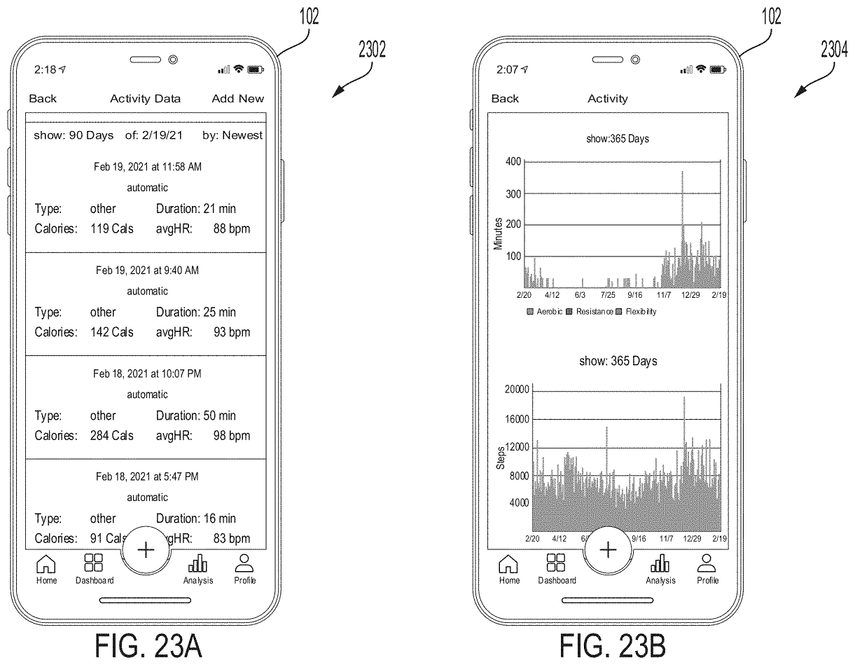

Additionally or alternatively to generating the recommendation notification message alerting the patient or the patient's clinician to the patient's deteriorating cardiovascular fitness, the application 110 and/or the diabetes management server 104 may generate example user interfaces 2302 and 2304 that chart all of the patient's workout data, such as that shown in FIGS. 23A and 23B. The user interface 2302 displays, on a display screen of the user device 102, statistics (e.g., workout type, duration, calories burned, average heart rate, number of steps, etc.) for every detected workout that the patient performed. The user interface 2304 displays, on the display screen of the user device 102, these statistics in the form of charts, or graphs, over various time periods (e.g., months, a year, etc.). The patient may visually see their workout performance with these charts, which may help encourage the patient to keep up the good performance or to improve poor performance. For instance, the patient may view the top chart that shows workout minutes over the course of the year and see that between about April 12 and July 25 they performed very little workouts.

In another example, if the application 110 and/or the diabetes management server 104 detects that the patient has been consistently performing workouts and correlates this with a decreasing resting heart rate baseline of the patient, the application 110 and/or the diabetes management server 104 can generate a recommendation notification message, based on an actionable recommendation 212, that congratulates the patient on lowering their resting heart rate. Generating a congratulatory recommendation notification message of this sort can help a patient see the benefits of performing workouts that they might not otherwise detect or that they might not detect as quickly.

As noted above, in some instances an elevated heart rate in heart rate data may not be a reliable indicator of a patient working out because the elevated heart rate may be due to stress rather than working out. In at least some aspects, the application 110 and/or the diabetes management server 104 distinguishes between a workout and stress by determining at least some of: whether a step count rate exceeds a predefined step rate threshold, whether a secondary factor indicative of the first lifestyle is present in the patient data 142, and whether the time period exceeds a predefined time threshold, as described above. The application 110 and/or the diabetes management server 104 can also be configured, in various aspects, to classify a time period as a period of stress or a lifestyle activity as a stress-causing activity.

FIG. 6 illustrates a flow diagram of an example procedure 600 for detecting that a patient is experiencing stress from the received patient data 142, according to an example embodiment of the present application. Although the procedure 600 is described with reference to the flow diagram illustrated in FIG. 6, it should be appreciated that many other methods of performing the steps associated with the procedure 600 may be used. For example, the order of many of the blocks may be changed, certain blocks may be combined with other blocks, and many of the blocks described may be optional. In an embodiment, the number of blocks may be changed. The actions described in the procedure 600 are specified by one or more instruction and may be performed among multiple devices including, for example, the application 110 on the user device 102 and/or the diabetes management server 104.

The example procedure 600 begins when patient data 142 including heart rate data for a patient is received in the application 110 and/or the diabetes management server 104 (block 602). The application 110 and/or the diabetes management server 104 then determines a resting heart rate baseline for the patient from the received heart rate data (block 604). The resting heart rate baseline for the patient is based on the patient's heart rate when the patient is inactive. For instance, the resting heart rate baseline can be an average value of the patient's inactive heart rates, a median value of the patient's inactive heart rates, or a distribution of all of the patient's inactive heart rates.

To obtain the patient's inactive heart rates from the received heart rate data, the application 110 and/or the diabetes management server 104 eliminates the heart rate data corresponding to periods during which the patient was active. For example, the application 110 and/or the diabetes management server 104 may eliminate heart rate data from periods that the application 110 and/or the diabetes management server 104 has classified (e.g., via the method 500) as the first lifestyle activity (e.g., a workout). In another example, the application 110 and/or the diabetes management server 104 may eliminate heart rate data from any period that includes step data. In another example, the application 110 and/or the diabetes management server 104 may eliminate heart rate data from any period during which the patient's location changed. In some aspects, the application 110 and/or the diabetes management server 104 may further eliminate heart rate data corresponding to a predefined period (e.g., 2 minutes, 5 minutes, etc.) directly prior to, and directly subsequent to, a period during which the patient was active.

In various embodiments, the application 110 and/or the diabetes management server 104 may determine the resting heart rate baseline from a set of the patient's inactive heart rates and then periodically update (e.g., every week, every day, every 5, 10, 30, 60 minutes, etc.) the resting heart baseline from subsequent sets of the patient's inactive heart rates. In other embodiments, the application 110 and/or the diabetes management server 104 may continually update the resting heart baseline as new inactive heart rates are received. The more often the resting heart baseline is updated, the more accurately the resting heart baseline will reflect any changes in the patient's lifestyle.

At some point after determining the resting heart rate baseline, the application 110 and/or the diabetes management server 104 may receive patient data 142 including step data and/or heart rate data over a time period (block 606). The application 110 and/or the diabetes management server 104 determine whether the time period is the first lifestyle activity (e.g., a workout) based on the received patient data 142 (block 608). For example, the application 110 and/or the diabetes management server 104 may perform blocks 504-514 of the example method 500 to determine whether the time period is the first lifestyle activity. If the application 110 and/or the diabetes management server 104 determines that the time period is the first lifestyle activity, then the application 110 and/or the diabetes management server 104 classifies the time period as the first lifestyle activity (block 610).

If the application 110 and/or the diabetes management server 104 determines that the time period is not the first lifestyle activity, then the application 110 and/or the diabetes management server 104 determines whether the heart rate data received at block 606 includes heart rates that exceed the resting heart rate baseline (block 612). As described above, the resting heart rate baseline may be an average or median of the patient's inactive heart rates. In such embodiments, the application 110 and/or the diabetes management server 104 determines whether the heart rate data received at block 606 includes heart rates that exceed the resting heart rate baseline by comparing an average or median of the heart rates in the heart rate data received at block 606 with the resting heart rate baseline. If the average or median of the heart rates in the heart rate data received a block 606 is less than or equal to the average or median heart rate of the resting heart rate baseline, then the application 110 and/or the diabetes management server 104 classifies the time period as a second lifestyle activity other than a stress-inducing activity (block 614). Stated differently, if the patient's average or median heart rate is not above the patient's resting heart rate baseline during the time period, then it is determined that the patient is not stressed during the time period.

If the average or median of the heart rates in the heart rate data received at block 606 is greater than the average or median heart rate in the resting heart rate baseline, then the application 110 and/or the diabetes management server 104 classifies the time period as a period of stress (block 616). In at least one embodiment, the average or median of the heart rates in the heart rate data received at block 606 must be greater than the average or median heart rate in the resting heart rate baseline by a threshold amount (e.g., a certain bpm, a certain percentage, etc.) for the application 110 and/or the diabetes management server 104 to classify the time period as a period of stress. The threshold amount may define a heart rate increase that corresponds to at least light stress. Such an embodiment may be beneficial in certain instances to avoid classifying a slight (e.g., 1 bpm) increase in heart rate from the resting heart rate baseline as a period of stress when it is, in fact, merely due to slight variation in the patient's resting heart rate throughout the day.

The application 110 and/or the diabetes management server 104 may then determine an overall intensity of the stress induced by the stress-inducing activity (block 618). In at least one embodiment, the overall intensity may be determined by comparing a difference between the average or median of the heart rates received at block 606 and the average or median heart rate of the resting heart rate baseline with a standard deviation of the inactive heart rates used to determine the resting heart rate baseline. For example, an overall intensity, or stress score, may be determined by Equation 1 below that utilizes average heart rates, in which σ is a standard deviation.

$$Stress Score of Time Period = \frac{\begin{matrix}avg ( time period ' s heart rates ) - \\ avg ( all inactive heart rates )\end{matrix}}{σ ( all inactive heart rates )} \qquad Equation 1$$

The standard deviation σ can be calculated by Equation 2 below in which xᵢ is a value of each inactive heart rate data point, μ is an average of all inactive heart rate data points, and N is a total count of data points.

$$σ = \sqrt{\frac{\sum {( {x}_{i} - μ )}^{2}}{N}} . \qquad Equation 2$$

Comparing the difference in heart rates from the resting heart rate baseline to a standard deviation rather than an absolute threshold helps eliminate differences between patients and/or times of the day (e.g., sleeping vs. working) that may otherwise affect the stress score determination. The application 110 and/or the diabetes management server 104 may define various ranges within which a stress score may fall to determine the overall intensity of stress. For instance, Table 1 below provides one example of such ranges for determining an overall intensity of the stress based on the determined stress score.

TABLE 1

| Stress Score (σ) | Stress Intensity |
|---|---|
| σ ≤ 0 | No stress |
| 0 < σ < 1 | Light stress |
| 1 ≤ σ < 2 | Moderate stress |
| σ ≥ 2 | High stress |

Once the intensity of the stress for the time period is determined, the application 110 and/or the diabetes management server 104 stores the time period with its stress intensity to a daily activity log of the patient as part of a health log and feedback routine. For example, the application 110 and/or the diabetes management server 104 may record that the patient was stressed at the determined stress intensity on the day and at the time corresponding to the time period. The patient and/or the patient's clinician can correlate this stress information with the lifestyle activity (e.g., eating, going to work, visiting a particular person or people, exercising, etc.) that the patient was performing during the stressful period, which can provide helpful insight into the patient's lifestyle activities. In some aspects, the application 110 and/or the diabetes management server 104 may query the patient for information about what the patient was doing during the stressful period and/or what the patient did just prior to or just after the stressful period. In other aspects, the application 110 and/or the diabetes management server 104 may utilize location information of the patient, a schedule information of the patient, or may otherwise detect the patient's activity (e.g., detecting the patient completed a workout) to correlate what the patient was doing just prior to, during, or after the stressful period. In this way, the application 110 and/or the diabetes management server 104 can determine an impact of external factors, environment, context, and/or activity on the patient's stress. The application 110 and/or the diabetes management server 104 may then return to collecting and analyzing patient data 142 for the next time period or event.

As described above, the example procedure 600 includes at least a portion of the blocks of the example procedure 500 at blocks 608 and 610. It should be appreciated that the example procedure 500 may similarly include at least a portion of the blocks of the example procedure 600. For example, blocks 602-604 may be added prior to block 502 and block 512 may be replaced by blocks 612-618. In such an example, after the application 110 and/or the diabetes management server 104 determines that the time period is not the first lifestyle activity at block 508 or block 510, the application 110 and/or the diabetes management server 104 determines whether the time period is a period of stress (e.g., the second lifestyle activity is a stress-inducing activity).

In various embodiments, the application 110 and/or the diabetes management server 104 may generate inactive heart rate data distributions (e.g., over hours of the day, days of the week, months of the year, etc.) that the user interface 702 may display on a display screen of the user device 102. The displayed inactive heart rate distributions can enable a time-correlated analysis of the patient's inactive heart rates. A patient or clinician can compare the patient's current inactive heart rate distributions with historical data of the patient's inactive heart rate distributions, such as comparing a certain day of the week across one or more months. The visualized data on the display screen of the user device 102 can help a patient or clinician appreciate the data in a different way than strictly numbers and therefore helps generate insights into the patient's lifestyle.

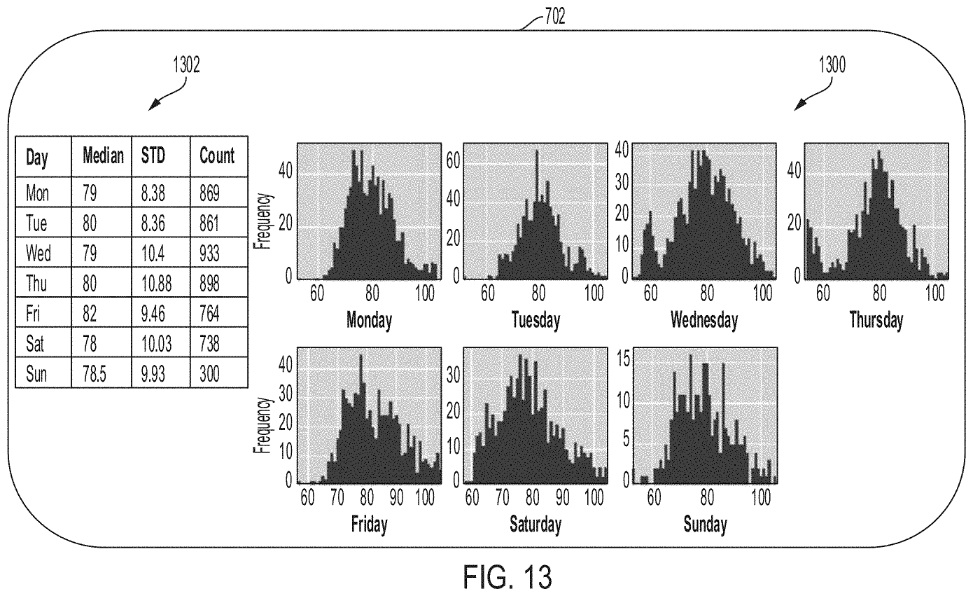

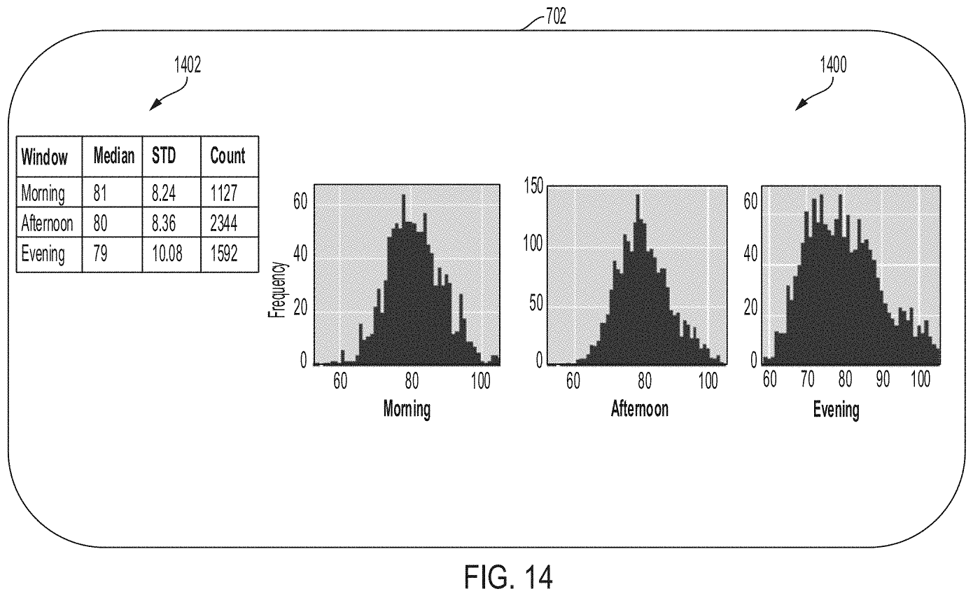

FIG. 13 illustrates the user interface 702 displaying, on a display screen of the user device 102, an example collection of inactive heart rate data distributions 1300 that each show a frequency of a patient's inactive heartrate at a particular beats per minute (bpm) value over the course of a day. The chart 1302 includes a median heart rate, a standard deviation, and a total count of heart rate values for each day of the week. In some aspects, the user interface 702 may display, on a display screen of the user device 102, one or more of the inactive heart rate distributions in the collection of inactive heart rate distributions 1300. FIG. 14 illustrates the user interface 702 displaying, on a display screen of the user device 102, an example collection of inactive heart rate data distributions 1400 that each show a frequency of a user's inactive heartrate at a particular bpm value at different parts (e.g., morning, afternoon, and evening) of a single day. The chart 1402 includes a median heart rate, a standard deviation, and a total count of heart rate values for each part of the day. In some aspects, the user interface 702 may display, on a display screen of the user device 102, one or more of the inactive heart rate distributions in the collection of inactive heart rate distributions 1400.

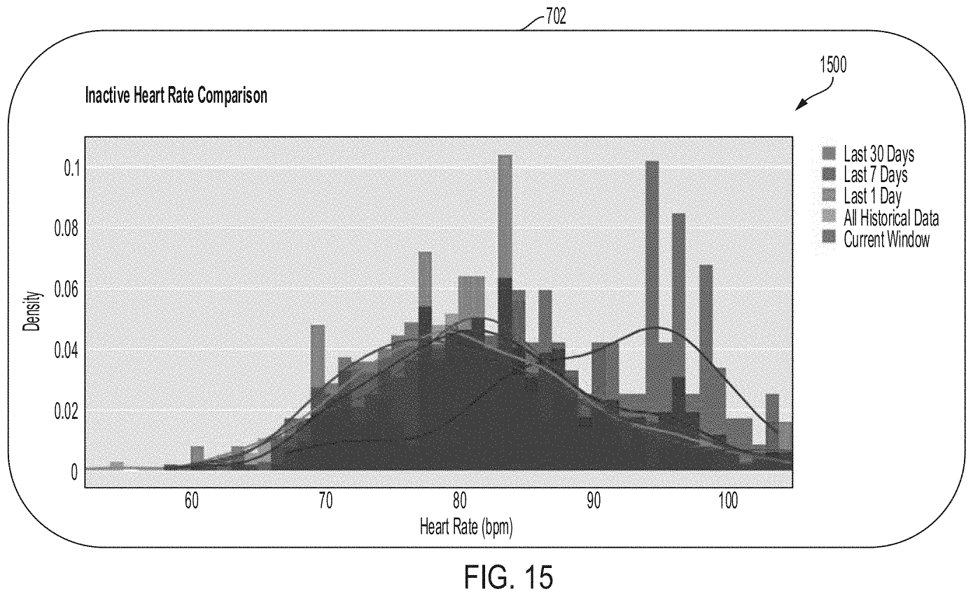

FIG. 15 illustrates the user interface 702 displaying, on a display screen of the user device 102, an example graph 1500 showing a comparison of a patient's heart rate data as a distribution over the past 1 day, past 7 days, and past 30 days. The graph 1500 enables a comparison of periods of interest against historical periods of interest. For example, if the application 110 and/or the diabetes management server 104 identifies that a median inactive heart rate is higher in the current 7 days versus the prior 7 days, or this month versus another month, the application 110 and/or the diabetes management server 104 may be able to deduce a difference in stress intensity, or level, based on that difference in median inactive heart rate.

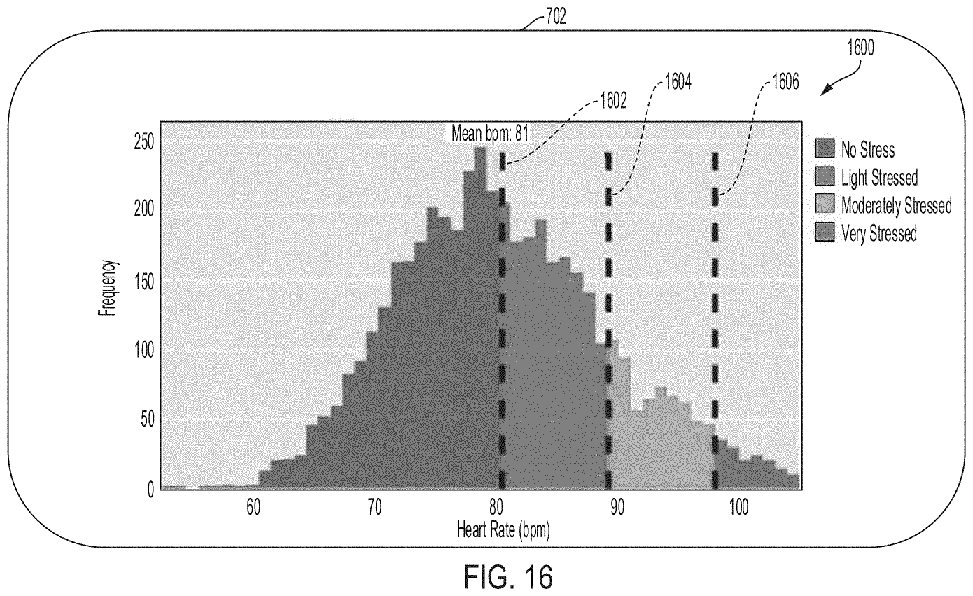

Similar to displaying inactive heart rate distributions, the application 110 and/or the diabetes management server 104 may generate stress score distributions (e.g., over minutes, hours, days, months, years, decades, etc.) that the user interface 702 may display on a display screen of the user device 102. FIG. 16 illustrates the user interface 702 displaying, on a display screen of the user device 102, an example graph 1600 showing a distribution of inactive heart rates over a single day with assigned stress scores. In this example, heart rates to the left of the dashed line 1602 correspond to periods of no stress, heart rates between the dashed line 1602 and the dashed line 1604 correspond to periods of light stress, heart rates between the dashed line 1604 and the dashed line 1606 correspond to periods of moderate stress, and heart rates to the right of the dashed line 1606 correspond to periods of high stress. In various instances, the different stress scores may be displayed as different colors. The graph 1600 enables visualizing the varying levels of stress that the patient experienced during the day as a whole.

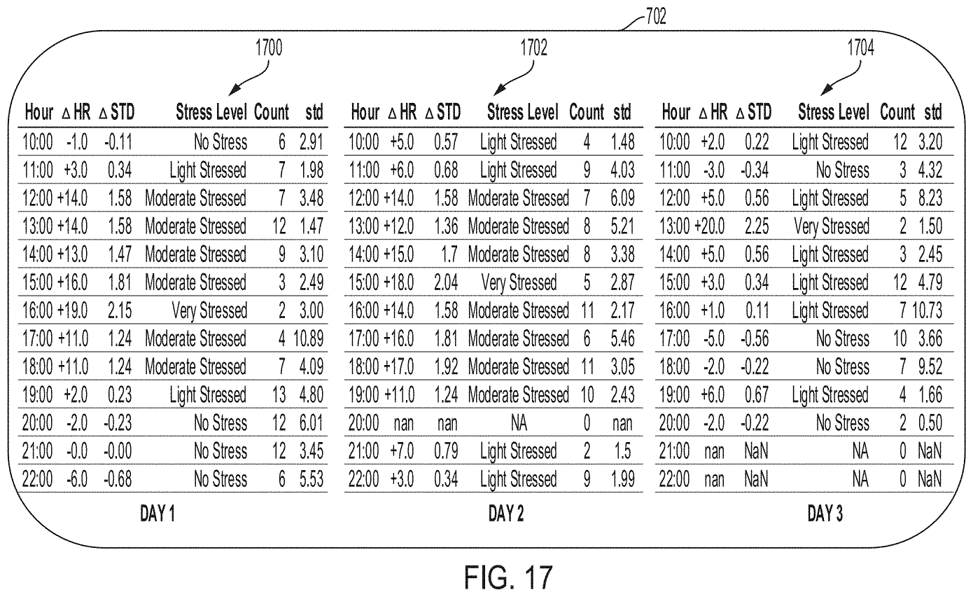

FIG. 17 illustrates the user interface 702 displaying, on a display screen of the user device 102, a collection of data tables showing a patient's stress score data distributed from 10 AM to 10 PM in one hour time blocks across a first day in table 1700, across a second day in table 1702, and across a third day in table 1704. In other examples, the time blocks may be any suitable time duration other than one hour. As shown in the tables 1700, 1702, and 1704, ΔHR is the difference between heart rate bpm in the time block and average historical inactive heart rate bpm, ΔSTD is the stress score, or the rate of ΔHR over the standard deviation (std) of historical inactive heart rate bpm, Stress Level is the intensity corresponding to the stress score of the time block, Count is the amount of data points available in the time block, and std is the standard deviation of the inactive heart rate bpm in the time block. In some instances, such as for 8 PM (i.e. 20:00) in table 1702, less than all of the time blocks may include a stress score if data was not collected for a certain time block. It should be appreciated that a patient or clinician can view any number of data tables distributing stress data across, e.g., minutes, hours, days, months, years, decades, etc. A patient or clinician can use the data tables to gain insight into how the patient's stress varies across the time blocks.

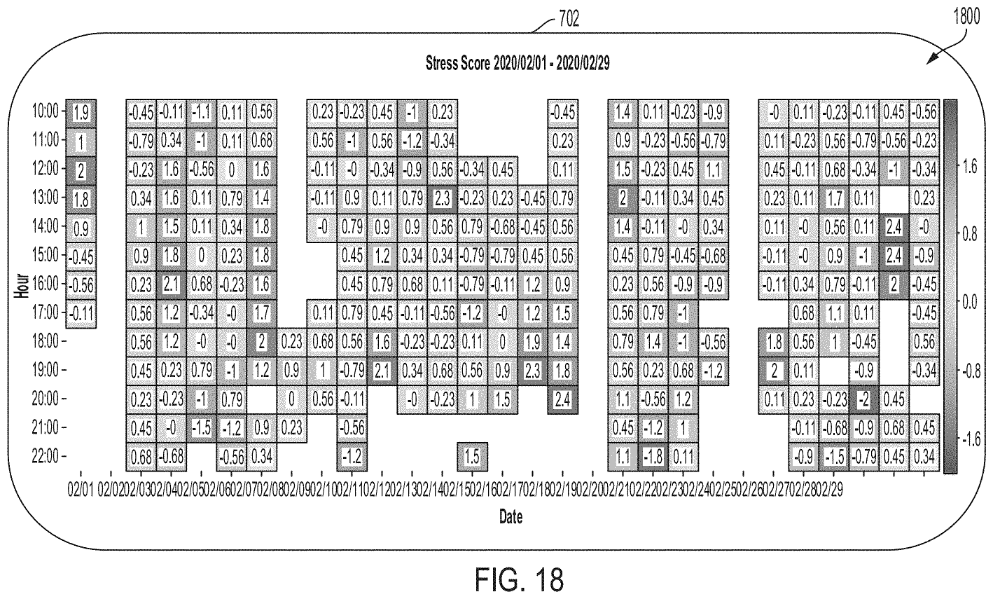

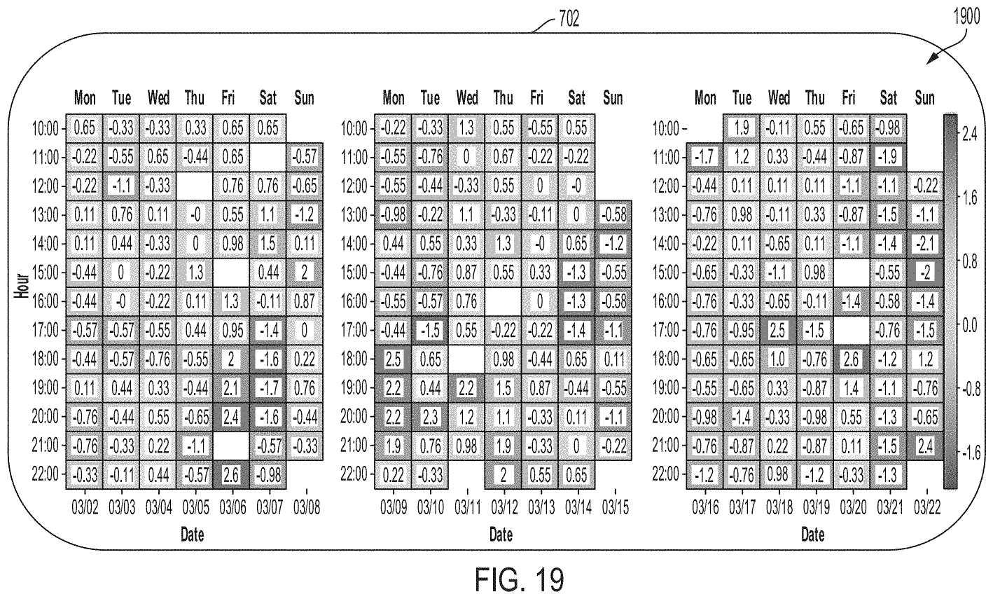

In some embodiments, the application 110 and/or the diabetes management server 104 may generate one or more stress heat maps from the stress data, such as from the stress data in data tables 1700, 1702, 1704. FIG. 18 illustrates the user interface 702 displaying, on a display screen of the user device 102, an example stress heat map 1800 that may be generated using stress score data. The stress heat map 1800 shows a patient's stress score data distributed from 10 AM to 10 PM across each date in February. In some embodiments, each block of time in the stress heat map 1800 may include the stress score corresponding to that block of time. In some embodiments, each block of time in the stress heat map 1800 may be color-coded corresponding to the stress intensity of the respective block of time. In some instances, such as for the example stress heat map 1800, less than all of the time blocks may include a stress score if data was not collected for a certain time block. The heat map 1800 can help the patient visualize stress trends and identify stressful periods of time. Similar to the heat map 1800, FIG. 19 illustrates the user interface 702 displaying, on a display screen of the user device 102, an example stress heat map 1900 that shows a patient's stress score data distributed from 10 AM to 10 PM across multiple dates in March that are further categorized as days of the week. This further categorization can help a patient visualize if certain days of the week are more stressful than others. A patient can also compare their stress levels in February with their stress levels in March.

In various embodiments, the application 110 and/or the diabetes management server 104 may provide a patient with various information that is related to, or is used to calculate, the stress score for a particular time block. For example, a patient may be particularly interested in information related to the stress scores of 02/04, 02/07, and 02/17 on the heat map 1800, which were three of the patient's most stressful days of February. The application 110 and/or the diabetes management server 104, in one example, could provide the patient with information regarding the activities performed by the patient or the locations visited by the patient on those days. Such information can provide guidance to a patient as to which activities and/or locations to avoid in order to reduce stress, or which activities and/or locations help provide a patient with stress release.

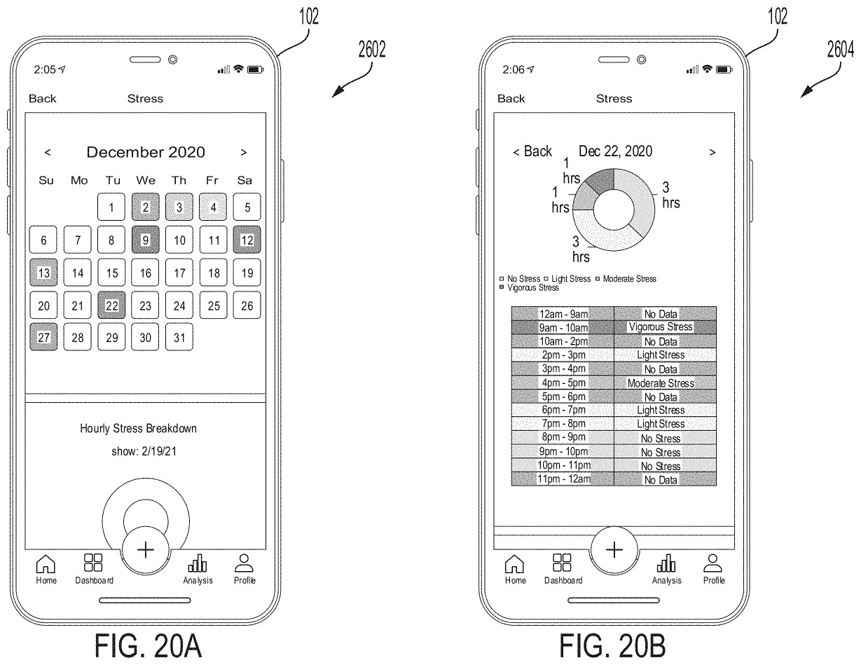

FIG. 20A illustrates a user interface 2602 displaying, on a display screen of the user device 102, a patient's stress data in the form of a calendar. In at least some aspects, each day on the calendar may be color-coded to correspond to a particular stress level, which can help the patient visualize the days that had the most stress during the calendar month. A patient may then select any one of the days to view an hourly breakdown of the patient's stress for that day, which the user interface 2604 may display on the display screen of the user device 102, as shown in FIG. 20B. The user interface 2604 can help the patient view how their stress levels varied throughout the day.

In some embodiments, the application 110 and/or the diabetes management server 104 may utilize the information related to stressful periods to provide active guidance to a patient to prevent a patient from performing stressful activities and/or navigating to stressful locations, or at least to notify the patient that they are performing a stressful activity and/or navigating to a stressful location so that the patient may be cognizant of their stress level. For example, the application 110 and/or the diabetes management server 104 may provide a recommendation notification message, based on an actionable recommendation 212, on the display screen of the user device 102 alerting the patient that the patient is approaching a stressful location.

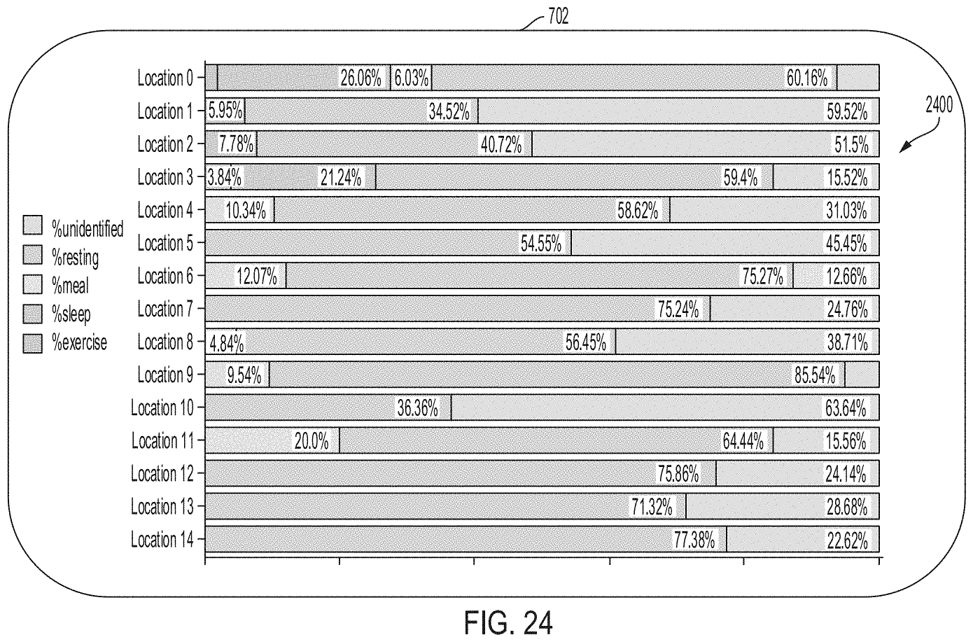

In addition to storing information on particular locations associated with stress for the patient, the application 110 and/or the diabetes management server 104 may associate various locations with different detected lifestyle activities of the patient. For instance, FIG. 24 illustrates the user interface 702 displaying, on a display screen of the user device 102, a graph 2400 that charts data indicating how the application 110 and/or the diabetes management server 104 has detected the patient spends their time at fourteen different locations. To collect this data, the application 110 and/or the diabetes management server 104 detects that a patient is at a particular location (e.g., Location 3), detects that the patient is performing a particular lifestyle activity (e.g., a workout), and correlates the detected location with the detected lifestyle activity. As the application 110 and/or the diabetes management server 104 collects more data, data on certain lifestyle activities may become more prominent than other activities at the particular location.

For example, the collected data indicates that when the patient is at Location 3, the patient exercises 3.84% of the time, sleeps 21.24% of the time, is in a resting state 59.4% of the time, and performs an unidentified activity (e.g., an activity that the application 110 and/or the diabetes management server 104 is unable to detect) 15.52% of the time. Location 3 could, for example, be the patient's home. In various embodiments, the application 110 and/or the diabetes management server 104 may detect sleep as the lifestyle activity of the patient based on the patient's heart rate data and/or movement data. For instance, the patient's heart rate can fall below the patient's resting heart rate baseline during sleep. In another example, the collected data indicates that when the patient is at Location 9, the patient is eating a meal 9.54% of the time, is in a resting state 85.54% of the time, and performs an unidentified activity the remaining percentage. Location 9 could, for example, be a restaurant. In various embodiments, the application 110 and/or the diabetes management server 104 may detect eating a meal as the lifestyle activity of the patient based on the patient's blood glucose data.

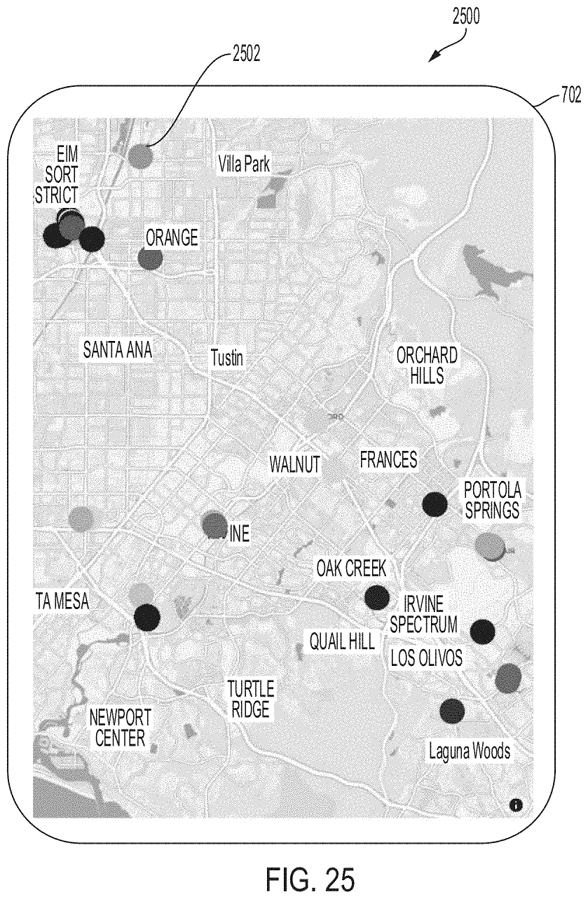

In various embodiments, the application 110 and/or the diabetes management server 104 may use the correlated location and lifestyle activity information to generate a map that includes indicators at the various locations. For example, FIG. 25 illustrates the user interface 702 displaying, on a display screen of the user device 102, an example map 2500 including example indicators 2502 at various locations. In various embodiments, the indicators 2502 may relate to any suitably relevant information pertaining to these locations. For example, the indicators 2502 may indicate the patient has experienced stress, or typically experiences stress, when at a location corresponding to the indicators 2502. In another example the indicators 2502 may indicate the patient has experienced a blood sugar spike, or typically experiences a blood sugar spike, when at a location corresponding to the indicators 2502. In either of these examples, the indicators 2502 may be color-coded to indicate a severity or intensity of the stress or blood sugar spike at a particular location.

The application 110 and/or the diabetes management server 104 may also correlate detected stress data for the patient with other patient information in order to provide active guidance to a patient regarding other aspects of the patient's life. For example, the application 110 and/or the diabetes management server 104 may correlate an increased number of stressful events for the patient with an increased resting heart rate baseline for the patient over the course of a few months and may generate a recommendation notification message, based on an actionable recommendation 212, suggesting the patient participate in some stress releasing activities. The patient may not have otherwise recognized that their resting heart rate baseline had increased and therefore, in this way, the application 110 and/or the diabetes management server 104 can provide helpful guidance to the patient as part of a holistic approach to improve the patient's diabetic health.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

1. A system for detecting lifestyle activities comprising:
a memory; and
a processor in communication with the memory, the processor configured to:
receive user data including step count data of a user and heart rate data of the user over a period of time;
determine at least one of: (i) a rate of steps in the received step count data is greater than a threshold step rate over at least a subset of the period of time, or (ii) the received heart rate data includes a heart rate above a threshold heart rate over at least a subset of the period of time;
determine whether a secondary factor indicative of a first lifestyle activity is present in the user data during the period of time;
when the secondary factor is not present, classify the period of time as a second lifestyle activity;
determine whether a length of the period of time is greater than a first threshold period of time when the secondary factor is present;
when the length of the period of time is less than the first threshold period of time, classify the period of time as the second lifestyle activity;
when the length of the period of time is greater than the first threshold period of time, classify the period of time as the first lifestyle activity; and
combine the period of time classified as the first lifestyle activity with a previous period of time classified as the first lifestyle activity in response to determining that less than a second threshold period of time has elapsed between the period of time classified as the first lifestyle activity and the previous period of time classified as the first lifestyle activity.

2. The system for detecting lifestyle activities of claim 1, wherein the step count data of the user and the heart rate data of the user is received from a fitness tracking device, an accelerometer of a user device, and a fitness tracking application operating on a user device.

3. The system for detecting lifestyle activities of claim 1, wherein the heart rate data is received at a lower sampling rate than the step count data is received.

4. The system for detecting lifestyle activities of claim 3, wherein the heart rate data is received at a sampling rate of between 5-10 minutes.

5. The system for detecting lifestyle activities of claim 1, wherein the threshold heart rate is equal to a percentage of a maximum heart rate of the user.

6. The system for detecting lifestyle activities of claim 5, wherein the percentage is in a range between 57-63%.

7. The system for detecting lifestyle activities of claim 1, wherein the secondary factor indicative of the first lifestyle activity is at least one of:
(i) a heart rate of the user exceeding the threshold heart rate when it is determined that the rate of steps is greater than the threshold step rate,
(ii) a location of the user being a location associated with the first lifestyle activity,
(iii) a rate of location change of the user exceeding a threshold rate, and
(iv) a step count greater than zero in the received step count data when it is determined that the received heart rate data includes a heart rate above the threshold heart rate.

8. The system for detecting lifestyle activities of claim 1, wherein the secondary factor indicative of the first lifestyle activity is a heart rate of the user exceeding the threshold heart rate when it is determined that the rate of steps is greater than the threshold step rate.

9. The system for detecting lifestyle activities of claim 1, wherein the secondary factor indicative of the first lifestyle activity is a location of the user being a location associated with the first lifestyle activity.

10. The system for detecting lifestyle activities of claim 1, wherein the secondary factor indicative of the first lifestyle activity is a rate of location change of the user exceeding a threshold rate.

11. The system for detecting lifestyle activities of claim 1, wherein the secondary factor indicative of the first lifestyle activity is a step count greater than zero in the received step count data when it is determined that the received heart rate data includes a heart rate above the threshold heart rate.

12. The system for detecting lifestyle activities of claim 1, wherein the first lifestyle activity is working out.

13. The system for detecting lifestyle activities of claim 1, wherein the second lifestyle activity is an activity other than working out.

14. The system for detecting lifestyle activities of claim 1, wherein the processor is further configured to categorize an intensity of the period of time classified as the first lifestyle activity based on at least one of:
(i) an average heart rate,
(ii) a median heart rate,
(iii) a maximum heart rate, and
(iv) a quantity of heart rate data points, of the received heart rate data in the period of time compared to a predetermined maximum heart rate up the user.

15. The system for detecting lifestyle activities of claim 1, wherein the processor is further configured to determine a resting heart rate baseline of the user based on previously received heart rate data of the user.

16. The system for detecting lifestyle activities of claim 15, wherein determining the resting heart rate baseline based on previously received heart rate data includes eliminating the heart rate data associated with periods of activity of the user from the previously received heart rate data.

17. The system for detecting lifestyle activities of claim 16, wherein heart rate data associated with a predetermined amount of time directly prior to, and directly subsequent to, the periods of activity of the user are further eliminated from the previously received heart rate data.

18. The system for detecting lifestyle activities of claim 15, wherein upon determining that the secondary factor is not present or that the length of the period of time is less than the first threshold period of time the processor is further configured to:
determine whether the received heart rate data includes an average or median heart rate that exceeds the determined resting heart rate baseline,
wherein when it is so determined, the second lifestyle activity is a stress-inducing activity, and
wherein when it is not so determined, the second lifestyle activity is an activity other than working out and other than a stress-inducing activity.

19. The system for detecting lifestyle activities of claim 18, wherein the processor is further configured to classify a severity of the stress induced by the stress-inducing activity based on a percentage of a standard deviation that an average of the received heart rate data is equal to.

20. A method for detecting lifestyle activities comprising:
receiving, from a fitness tracking device, an accelerometer of a user device, or a fitness tracking application operating on a user device, user data including step count data of a user and heart rate data of the user over a period of time;
determining at least one of:
(i) a rate of steps in the received step count data is greater than a threshold step rate over at least a subset of the period of time, or
(ii) the received heart rate data includes a heart rate above a threshold heart rate over at least a subset of the period of time;
determining whether a secondary factor indicative of a first lifestyle activity is present in the user data during the period of time;
when the secondary factor is not present, classify the period of time as a second lifestyle activity;
determining whether a length of the period of time is greater than a first threshold period of time when the secondary factor is present;
when the length of the period of time is less than the first threshold period of time, classify the period of time as the second lifestyle activity;
when the length of the period of time is greater than the first threshold period of time, classify the period of time as the first lifestyle activity; and
combining the period of time classified as the first lifestyle activity with a previous period of time classified as the first lifestyle activity in response to determining that less than a second threshold period of time has elapsed between the period of time classified as the first lifestyle activity and the previous period of time classified as the first lifestyle activity.

21. The method for detecting lifestyle activities of claim 20, wherein determining whether the length of the period of time is greater than the threshold period of time includes determining a termination point of the period of time.

22. The method for detecting lifestyle activities of claim 21, wherein determining the termination point of the period of time includes at least one of:
(i) determining that the rate of steps in the received step count data is no longer greater than the threshold step rate,
(ii) determining that the received heart rate data no longer includes a heart rate above the threshold heart rate,
(iii) receiving location data indicating that the user has left the location associated with the first lifestyle activity, and
(iv) receiving location data indicating that the rate of location change of the user no longer exceeds the threshold rate.

23. The method for detecting lifestyle activities of claim 21, wherein determining the termination point of the period of time includes determining that the rate of steps in the received step count data is no longer greater than the threshold step rate.

24. The method for detecting lifestyle activities of claim 21, wherein determining the termination point of the period of time includes determining that the received heart rate data no longer includes a heart rate above the threshold heart rate.

25. The method for detecting lifestyle activities of claim 21, wherein determining the termination point of the period of time includes receiving location data indicating that the user has left the location associated with the first lifestyle activity.

26. The method for detecting lifestyle activities of claim 21, wherein determining the termination point of the period of time includes receiving location data indicating that the rate of location change of the user no longer exceeds the threshold rate.

27. The method for detecting lifestyle activities of claim 21, wherein determining the termination point includes interpolating between a last heart rate data point that exceeds the threshold heart rate and a first heart rate data point that fails to exceed the threshold heart rate.

28. The method for detecting lifestyle activities of claim 20, wherein determining a start of the period of time includes interpolating between a last heart rate data point that fails to exceed the threshold heart rate and a first heart rate data point that exceeds the threshold heart rate.
